(19) United States
(12) Patent Application Publication
(10) Pub. No.: US 2026/0199523 A1
(43) Pub. Date: Jul. 16, 2026

(54) NANOPARTICLES AND METHODS FOR MAKING THE SAME AND THEIR USE

(71) Applicant: UNIVERSITY OF PITTSBURGH-OF THE COMMONWEALTH SYSTEM OF HIGHER EDUCATION, Pittsburgh, PA (US)

(72) Inventors: Or SHEMESH, Pittsburgh, PA (US); Krishnashis CHATTERJEE, Pittsburgh, PA (US); Pururav RAMAKRISHNA, Pittsburgh, PA (US)

(21) Appl. No.: 19/134,002

(22) PCT Filed: Nov. 29, 2023
(86) PCT No.: PCT/US2023/081569

§ 371 Date: May 29, 2025

Related U.S. Application Data
(60) Provisional application No. 63/385,297, filed on Nov. 29, 2022.

(51) Int. Cl. A61K 48/00; A61K 33/242; A61K 33/38; A61K 41/00; A61P 35/00; C07K 14/195; C07K 14/435; C12N 9/02; C12N 9/08; C12N 15/85

(52) U.S. Cl. CPC A61K 48/005; A61K 33/242; A61K 33/38; A61K 41/0052; A61P 35/00; C07K 14/195; C07K 14/43586; C12N 9/0061; C12N 9/0065; C12N 15/85; C12Y 110/03002; C12Y 111/01014

(57) ABSTRACT

The present disclosure relates to nanoparticles, methods of making a nanoparticle in a cell, and uses thereof.

### RELATED APPLICATION

This PCT application claims priority to, and the benefit of, U.S. Provisional Patent Application No. 63/385,297, filed Nov. 29, 2022, which is incorporated by reference herein in its entirety.

### REFERENCE TO SEQUENCE LISTING

The sequence listing submitted on Nov. 29, 2023, as an .XML file entitled “10504-088WO1_ST26.xml” created on Nov. 29, 2023, and having a file size of 41,737 bytes is hereby incorporated by reference pursuant to 37 C.F.R. § 1.52(e)(5).

### FIELD

The present disclosure relates to a method of making and/or using a nanoparticle comprising a metal.

### BACKGROUND

In biomedicine, genetically encoded compounds, which include proteins and nucleic acids, are often used as diagnostic and therapeutic agents. Nanoparticles emerged as a class of compounds that can expand the applications of genetically encoded compounds in detecting, treating, and reversing disease. This is because nanoparticles can withstand and react with physical conditions such as heat or radiation at scales to which proteins, lipids, and nucleic acids are not able. However, using nanoparticles in living cells is challenging, as they tend to aggregate and often fail to reach the target cell or subcellular structure. These problems hinder the widespread use of nanoparticles in biology and medicine.

Given the limitations described above, there is a need to develop strategies to improve nanoparticles assembly and delivery to cellular and subcellular targets.

### SUMMARY

The present disclosure provides nanoparticle compositions and methods of making, preparing, generating, and/or forming said compositions. In some embodiments, the nanoparticle compositions are used for treating a cancer, or a microbial infection or treating or preventing radiation damage.

In some aspect, disclosed herein is a method of making a nanoparticle comprising one or more metals in a cell, the method comprising expressing a nucleic acid sequence encoding one or more metal binding peptides and one or more metal reducing peptides in the cell, administering a composition comprising an ion of each of the one or more metals, or a salt thereof, to the cell, and forming the nanoparticle within the cell upon reduction of the ion of each of the one or more metals.

In some embodiments, the one or more metals are selected from the group consisting of gold, silver, zinc, titanium, lead and potassium. In some embodiments, the composition comprises the ion of each of the one or more metals is selected from a group consisting of chloroauric acid (HAuCl₄), silver nitrate (AgNO₃), zinc oxide (ZnO), titanium dioxide (TiO₂), lead sulfide (PbS), potassium tetrachloroplatinate (K₂PtCl₄), or any salt thereof.

In some embodiments, one of the one or more metals is gold and the composition comprising the ion is chloroauric acid (HAuCl₄). In some embodiments, the one or more gold metal binding peptides comprise a sequence selected from the group consisting of SEQ ID NO:1, SEQ ID NO:2, and SEQ ID NO: 28. In some embodiments, the one or more gold metal reducing peptides are selected from the group consisting of WW, WWW, SEQ ID NO:3 (WWWW), SEQ ID NO:4 (WWWWW), SEQ ID NO:5 (WWWWWW), and SEQ ID NO: 6 (WWWWWWW).

In some embodiments, one of the one or more metals is silver and the composition comprising the ion is silver nitrate (AgNO₃). In some embodiments, the one or more silver metal binding peptides and the one or more silver metal reducing peptides comprise a sequence selected from the group consisting of SEQ ID NO:7 and SEQ ID NO: 27.

In some embodiments, the nucleic acid sequence encodes two or more copies of SEQ ID NO: 1, SEQ ID NO:2, SEQ ID NO:7, SEQ ID NO: 27, and/or SEQ ID NO: 28. In some embodiments, an expression vector comprises the nucleic acid sequence. In some embodiments, the nucleic acid sequence further comprises one or more linker encoding sequences. In some embodiments, the linker comprises SEQ ID NO:8, or a fragment thereof.

In some embodiments, the nucleic acid sequence further comprises a sequence encoding one or more cage-like peptides, or a fragment thereof. In some embodiments, the one or more cage-like peptides are selected from the group consisting of apoferritin, encapsulin, sericin, laccase, and ligninase. In some embodiments, the one cage-like peptide is an encapsulin peptide, and the nucleic acid sequence comprises one or more fragments of the encapsulin peptide.

In some embodiments, the method further comprises exposing the cell to radiation, and wherein radiation damage in the cell is reduced compared to a control. In some embodiments, the cell is in a subject and the radiation is x-rays, gamma rays, electron beams, protons, or combinations thereof.

In some embodiments, the cell is a cancer cell in a subject and the method further comprises administering a therapeutically effective amount of an electromagnetic radiation to the cancer cell, and wherein the method treats the cancer.

In some embodiments, the cell is in a subject and the method further comprises detecting the location of the nanoparticle in the subject using an imaging method selected from computed tomography (CT), positron emission tomography (PET), magnetic resonance imaging (MRI), and variations thereof.

In some embodiments, the cell is in a subject, the subject has a microbial infection, and wherein the method treats the microbial infection.

In one aspect, disclosed herein is a nanoparticle made by the method of any preceding or following aspect.

In another aspect, disclosed herein is a nanoparticle composition comprising a metal, one or more metal binding peptides and one or more metal reducing peptides. In some embodiments, the composition comprises the one or more metals selected from the group consisting of gold, silver, zinc, titanium, lead and potassium. In some embodiments, one of the one or more metals is gold. In some embodiments, the composition comprises the one or more gold metal binding peptides comprise a sequence selected from the group consisting of SEQ ID NO:1 SEQ ID NO:2, and SEQ ID NO: 28.

In some embodiments, one or more gold metal reducing peptides are selected from the group consisting of WW, WWW, SEQ ID NO:3 (WWWW), SEQ ID NO:4 (WWWWW), SEQ ID NO:5 (WWWWWW), and SEQ ID NO: 6 (WWWWWWW).

In some embodiments, one of the one or more metals is silver. In some embodiments, the one or more silver metal binding peptides and the one or more silver metal reducing peptides comprise SEQ ID NO:8. In some embodiments, the composition comprises two or more copies of SEQ ID NO: 1, SEQ ID NO:2, SEQ ID NO:7, SEQ ID NO: 27, and/or SEQ ID NO: 28.

In some embodiments, the composition further comprises one or more linkers. In some embodiments, the linker comprises SEQ ID NO:8, or a fragment thereof. In some embodiments, the composition further comprises one or more cage-like peptides, or a fragment thereof.

### BRIEF DESCRIPTION OF FIGURES

The accompanying figures, which are incorporated in and constitute a part of this specification, illustrate several aspects described below.

FIG. 1(A-D) shows the fluorescence microscope images of HeLa cells only (1A), HeLa cells transfected with GFP-3WAuBP1 (1B), HeLa cells treated with 1 mM Tetrachloroauric acid (HAuCl4) (1C), and HeLa cells transfected with plasmid encoding gold binding and reducing peptide GFP-3WAuBP1 and subsequent treatment with 1 mM Tetrachloroauric acid (HAuCl₄) (1D).

FIG. 2(A-D) shows the fluorescence microscope images of HeLa cells only (2A), HeLa cells transfected with GFP-Midas2 (2B), HeLa cells treated with 1 mM Tetrachloroauric acid (HAuCl₄) (2C), and HeLa cells transfected with plasmid encoding gold binding and reducing peptide GFP-GFP-Midas2 and subsequent treatment with 1 mM Tetrachloroauric acid (HAuCl₄) (2D).

FIG. 3 shows the Transmission Electron Microscope images of HeLa cells only without transfection with plasmids or addition of HAuCl₄.

FIG. 4 shows the Transmission Electron Microscope images of HeLa cells transfected with GFP-3WAuBP1 encoding control plasmid without addition of HAuCl₄.

FIG. 5 shows the Transmission Electron Microscope images of HeLa cells transfected with GFP-Midas2 encoding plasmid without addition of HAuCl₄.

FIG. 6 shows the Transmission Electron Microscope images of HeLa cells treated with HAuCl₄ without any transfection. Circles indicate gold nanoparticles in cytoplasm of salt (HAuCl₄) only control.

FIG. 7 shows the Transmission Electron Microscope images of HeLa cells transfected with plasmid encoding GFP-3WAuBP1 and subsequently treated with 1 mM HAuCl₄. Circles indicate area with more gold nanoparticles in cytoplasm than control.

FIG. 8 shows the Transmission Electron Microscope images of HeLa cells transfected with plasmid encoding GFP-Midas2 and subsequently treated with 1 mM HAuCl₄. Circles enclose area with more gold nanoparticles in cytoplasm than control.

FIG. 9(A-D) shows the fluorescence microscope images of HMC3 (microglia) cells only (9A), microglia cells transfected with GFP-3WAuBP1 (9B), microglia cells treated with 1 mM Tetrachloroauric acid (HAuCl₄) (9C), and microglia cells transfected with plasmid encoding gold binding and reducing peptide GFP-3WAuBP1 and subsequent treatment with 1 mM HAuCl4 (9D).

FIG. 10(A-D) shows the fluorescence microscope images of HMC3 (microglia) cells only (10A), microglia cells transfected with GFP-Midas2 plasmid (10B), microglia cells treated with 1 mM Tetrachloroauric acid (HAuCl₄) (10C) and microglia cells synthesized by transfecting cells with plasmid encoding gold binding and reducing peptide GFP-Midas2 and subsequent treatment with 1 mM HAuCl₄ (10D).

FIG. 11A is a schematic showing the engineering of genetically encoded nanoparticles in cells. FIG. 11B shows GFP ‘puncta’ expression on the outer surface of the protein cages showing the intracellular co-localization.

FIG. 12A is a schematic showing the targeting genetically encoded nanoparticles to organelles. FIG. 12B shows expression of GFP tagged with mitochondria localization sequence.

FIG. 13A shows confocal imaging and FIG. 13B shows TEM imaging of HeLa cells expressing Thermotoga maritima Encapsulin (15 nm)-GFP. FIG. 13B Scale bar=100 nm.

FIG. 14A shows confocal imaging and FIG. 14B shows TEM imaging of HeLa cells expressing Myxococcus xanthus Encapsulin (30 nm)-GFP. FIG. 14B Scale bar=100 nm.

FIG. 15 shows a confocal image, reflectance mode, of gold nanoparticles. This image is a proof of concept that shows that reflectance mode confocal could be used to image gold nanoparticles in HeLa cells.

FIG. 16(A-B) shows schematics of the metal nanoparticles as genetically encoded EM contrast agents (multi-tag EM (16A); live EM (16B)).

FIG. 17(A-B) shows confocal imaging (17A) and TEM imaging (17B) of genetically encoded silver nanoparticles. FIG. 17 shows the confocal image for expression of GFP tagged with protein cages containing silver nanoparticles and transmission electron microscopy image of the same protein cages containing electron dense silver nanoparticles.

FIG. 18(A-G) shows a schematic and data relating to genetically encoded nanoparticles (GENPs) synthesized intracellularly. FIG. 18A is a schematic of intracellular synthesis of genetically encoded gold nanoparticles. FIG. 18B shows HeLa cells expressing GFP tagged metal reducing proteins. FIG. 18C shows optical microscopy showing GENP formation in HeLa cells. FIG. 18D shows electron microscopy showing GENP formation. FIG. 18E shows characterization of GENPs for various peptides and salt concentrations. FIG. 18F shows elemental analyses of intracellular GENPs by Energy Dispersive X Ray Spectroscopy. FIG. 18G (left) shows the electron dense and control areas in the cell that were used to generate the corresponding EDX spectra (right) showing elemental characterization of intracellular gold nanoparticles.

FIG. 19(A-G) shows HeLa cells exposed to multiple concentrations of HAuCl₄ in PBS. FIG. 19A shows HeLa cells exposed to increasing concentrations of HAuCl₄. FIG. 19B shows HeLa cells expressing Midas2 exposed to multiple concentrations of HAuCl₄ in PBS. FIG. 19C shows HeLa cells expressing Midas2 exposed to multiple concentrations of HAuCl₄ in growth media. FIG. 19D shows TEM images of HeLa cells expressing Midas2 and exposed to 100 μM HAuCl₄ in growth media. FIG. 19E shows a viability graph corresponding to FIG. 19A with circles representing PBS only treatment, squares indicating 10 μM HAuCl4 in PBS treatment, triangles indicating 50 μM HAuCl₄ in PBS treatment, inverted triangles represent 100 μM HAuCl₄ in PBS treatment. FIG. 19F shows a viability graph corresponding to FIG. 19B with circles representing PBS only treatment, squares indicating 10 μM HAuCl₄ in PBS treatment, triangles indicating 50 μM HAuCl₄ in PBS treatment, inverted triangles represent 100 μM HAuCl4 in PBS treatment. FIG. 19G shows a viability graph corresponding to FIG. 19C with circles representing PBS only treatment, squares indicating 10 μM HAuCl4 in PBS treatment, triangles indicating 50 μM HAuCl₄ in PBS treatment, inverted triangles represent 100 μM HAuCl₄ in PBS treatment.

FIG. 20(A-C) shows images of GL261 glioblastoma cells expressing D4 or Gold Necklace peptide and exposed to 100 μM HAuCl₄ in growth media before and after laser treatment at 15 W/cm2 for 15 minutes. Top row: Cell only controls Second row: Cells transfected with Gold Necklace peptide only. Third row: Cells exposed to HAuCl₄ only. Bottom row: Cells transfected with Gold Necklace peptide and exposed to HAuCl₄. FIG. 20B is a graph showing number of dead cells before and after the laser treatment, with the first two bars being cells only, the third and fourth bars being D4 only, the fifth and sixth bars being HAuCl₄ only, and the seventh and eighth bars being D4 and HAuCl₄. FIG. 20C is a graph showing difference in the number of dead cells for each condition after the laser treatment.

FIG. 21(A-C) shows TEM images of GENPs. FIG. 21A shows GENPs in GL261 cells with 100 μM HAuCl₄ in growth media (top left), TEM images of GL261 cells transfected with Midas2 peptide and exposed to 100 μM HAuCl₄ (top right), TEM images of HEPA 1-6 cells exposed to 100 μM HAuCl₄ in growth media (bottom left), and TEM images of HEPA 1-6 cells transfected with Midas2 peptide and exposed to 100 μM HAuCl₄ in growth media (bottom right). FIG. 21B shows Gold Flake formation in HEPA 1-6 cells (top) and zoomed in images of gold flakes in HEPA1-6 cells (bottom). FIG. 21C shows TEM images of GENP formation in mice brain with Gold Necklace peptide and 1 mM HAuCl₄ (right panel corresponds to magnified image of white box). Peptide expression in mice brain is shown in inset.

FIG. 22(A-F) shows bioluminescent images of tumors in the brain of live mice and the graphs showing the variation of body weights of the mice and signal intensity from luciferase expressing tumor cells which were used as metrics for monitoring the health and the tumor size of the mice during the entire duration of the laser therapy experiments. FIG. 22(A-B) are each a representative stack of bioluminescence images of tumor in brains of control mice before and after in-vivo photothermal therapy respectively. FIGS. 22C and 22D each show a representative stack of bioluminescence images of tumors in mice brains treated with Midas2 peptide and exposed to 1 mM HAuCl₄, before and after in-vivo photothermal therapy, respectively. FIG. 22E is a graph showing change in weights of the mice in different categories for the entire duration of the photothermal therapy experiment. FIG. 22F is a graph showing the BLI signals in brains of mice in different categories for the entire duration of the experiment. FIGS. 22 E and F contains the following color scheme for various conditions and controls that were used for the photothermal therapy experiments, Blue: Mice with tumors treated with 1 mM HAuCl₄ and subsequent laser treatment, Cyan: Mice with tumor that didn't receive any laser treatment, Black: Mice with tumors transfected with D4 plasmid followed by 1 mM HAuCl₄ addition and subsequent laser treatment, Green: Mice with tumors transfected with Midas2 plasmid followed by 1 mM HAuCl4 addition and subsequent laser treatment and Red: Mice with tumor that received laser treatment.

FIG. 23(A-B) shows a schematic of genetically encoded nanoparticles used for CT Imaging.

FIG. 24(A-D) shows In-vitro testing of CT contrast properties of gold GENPs in HeLa cells using microCT. (A) (Top to bottom) CT scans of PCR tubes containing HeLa cells only, HeLa cells expressing gold necklace peptide, HeLa cells expressing gold necklace peptide incubated with iodine contrast agent, and HeLa cells expressing gold necklace peptide incubated with HAuCl₄ and producing gold GENPs, suspended in DI water. (B) Line graph of average gray values in the PCR tube from left to right. Arrow pointing to peak at the border between solution and pellet of HeLa cells expressing gold GENPs (HeLa+Gold necklace peptide+HAuCl4). (C&D) Cross-section analysis of CT scans showing bright contrast of HeLa cells expressing gold GENPs. (C) Full CT scans labeled with cross-sectional layers. (D) Corresponding cross-section slices. (Top) HeLa cells expressing gold necklace peptide incubated with iodine contrast agent, and (Bottom) HeLa cells expressing gold necklace peptide incubated with HAuCl₄ and producing gold GENPs. (1) Cross sections of the solution above cell pellet, (2) border of solution and cell pellet, (3) ½ depth in the cell pellet, and (4) ¾ depth in the cell pellet.

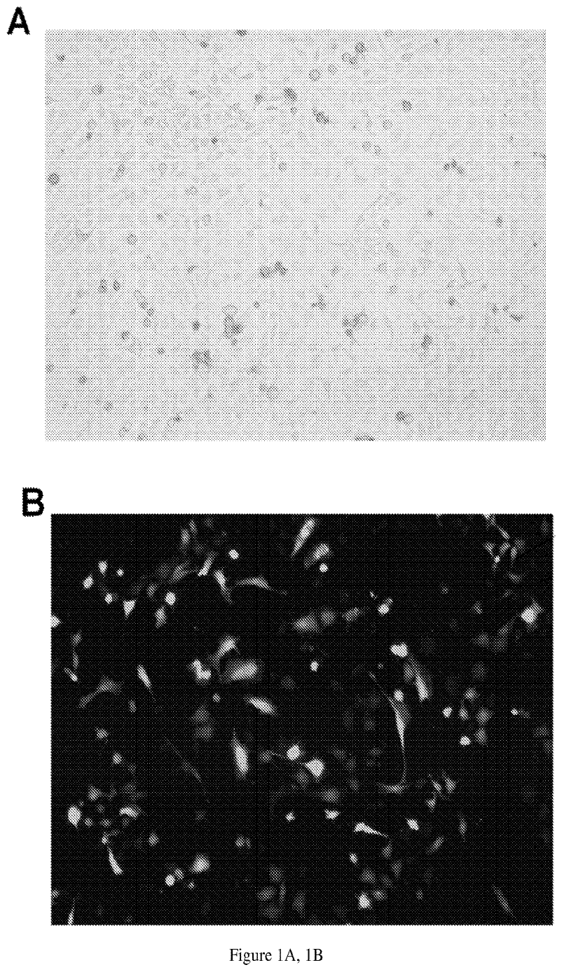

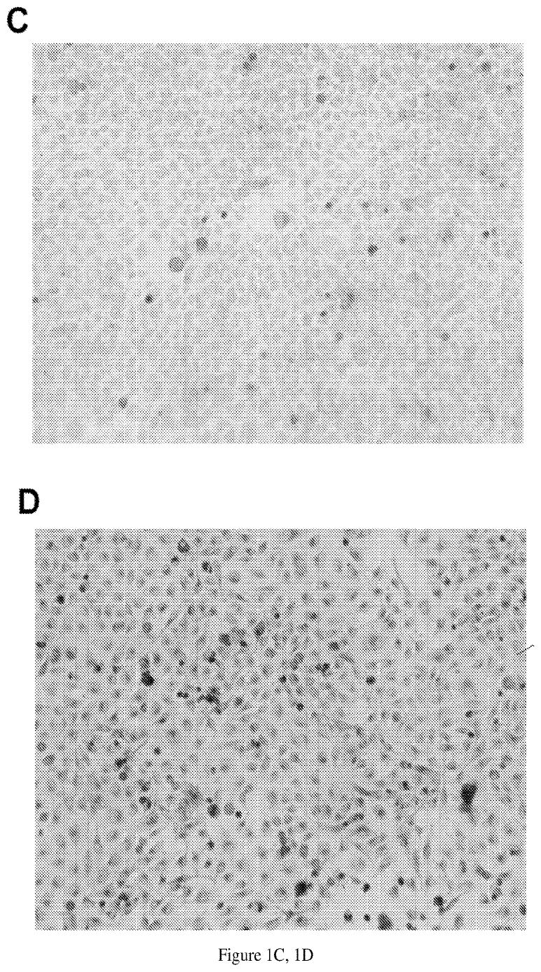

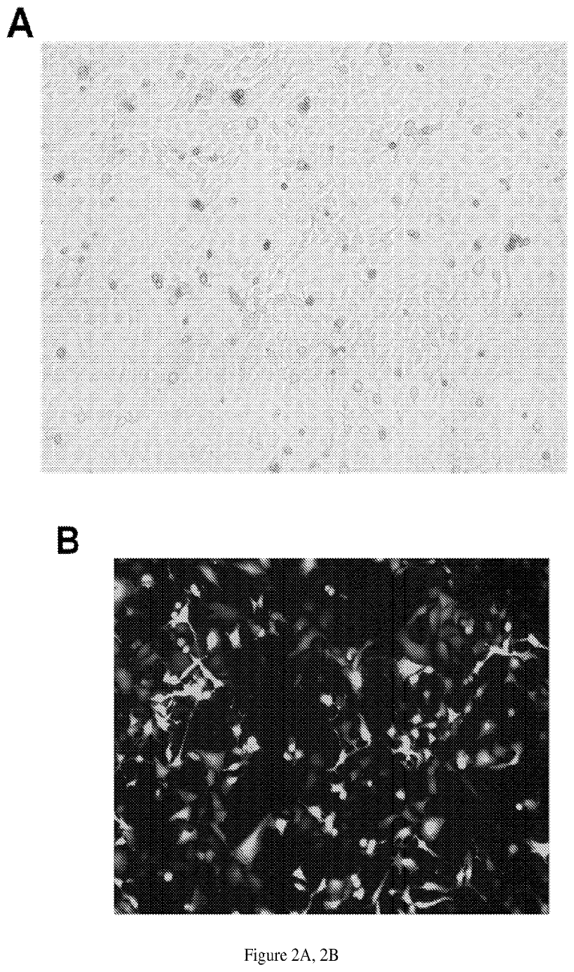

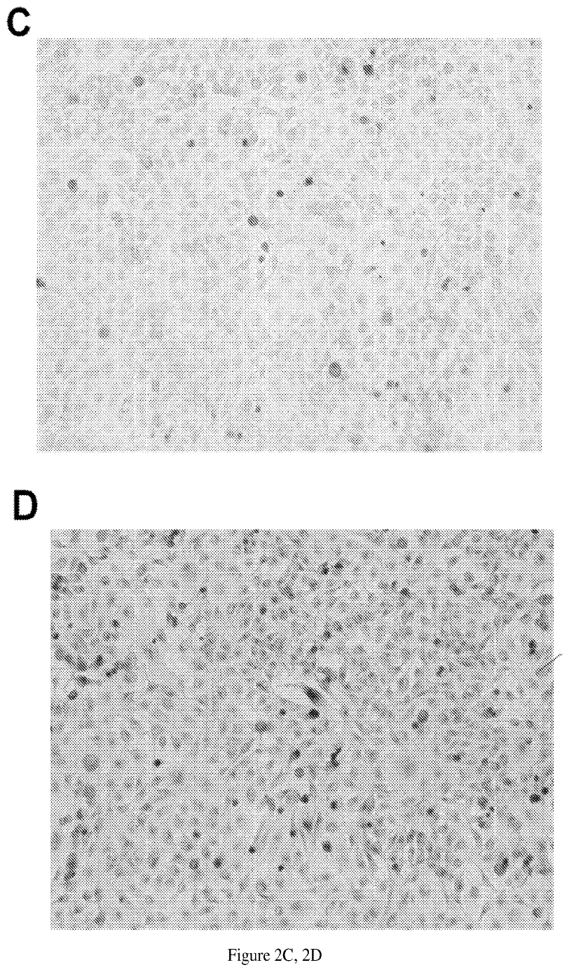

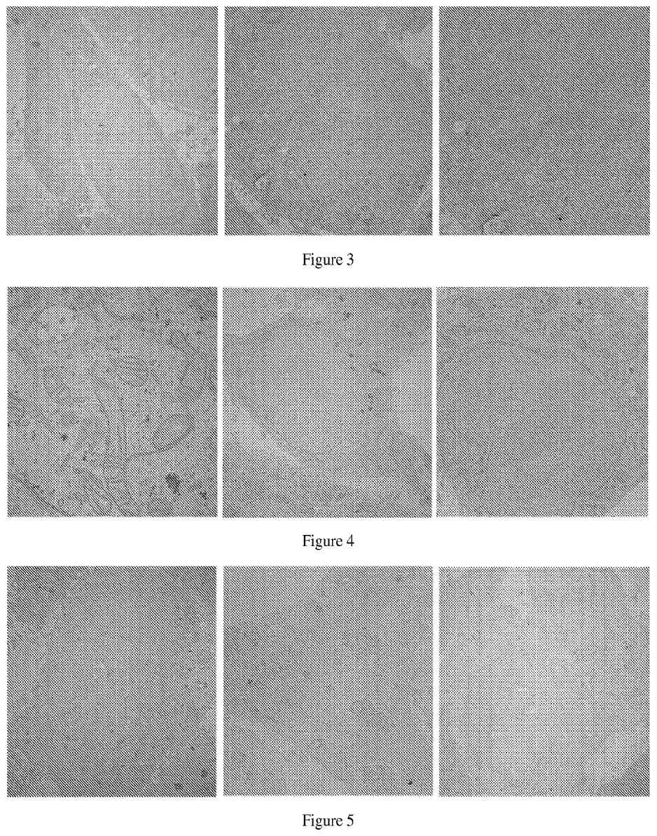

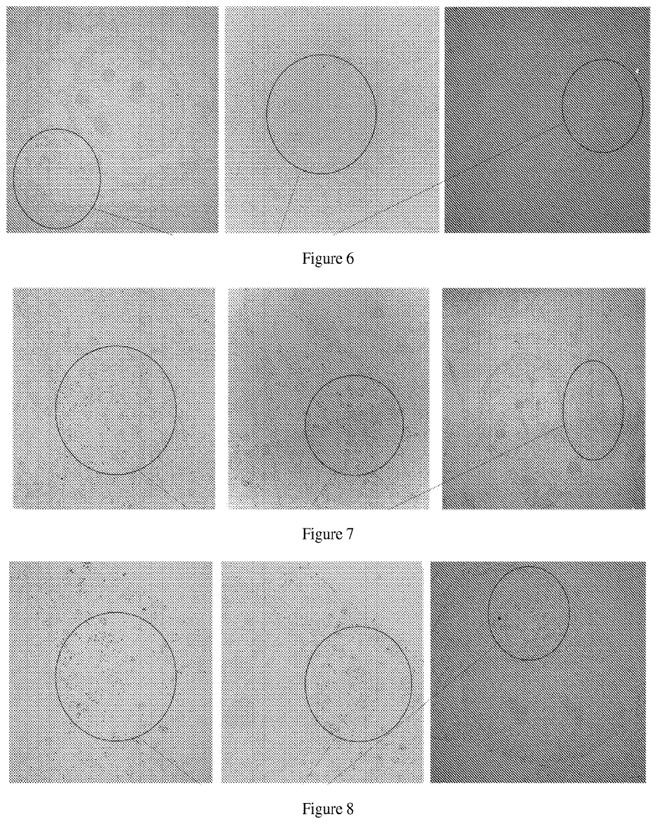

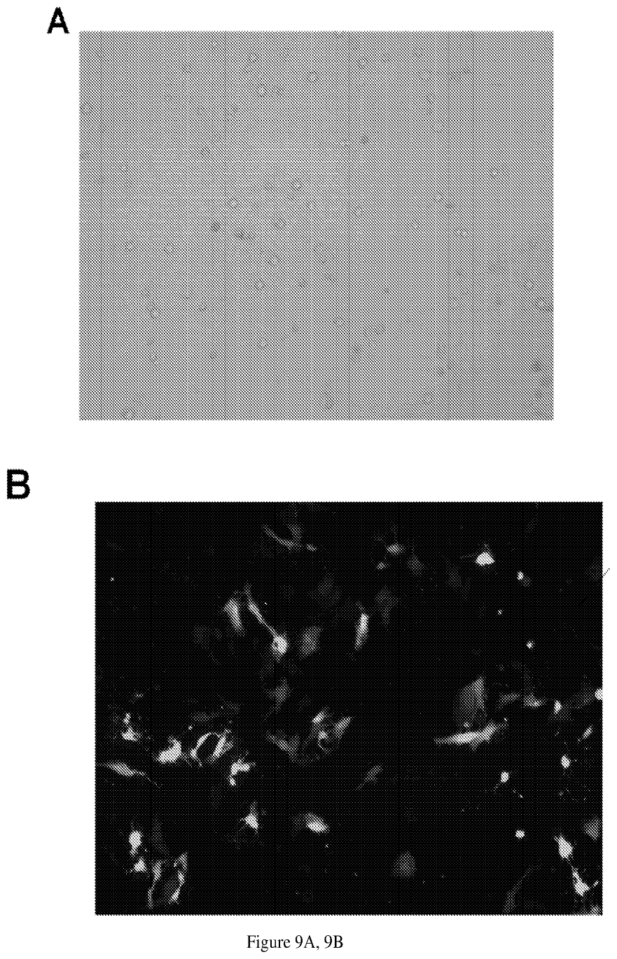

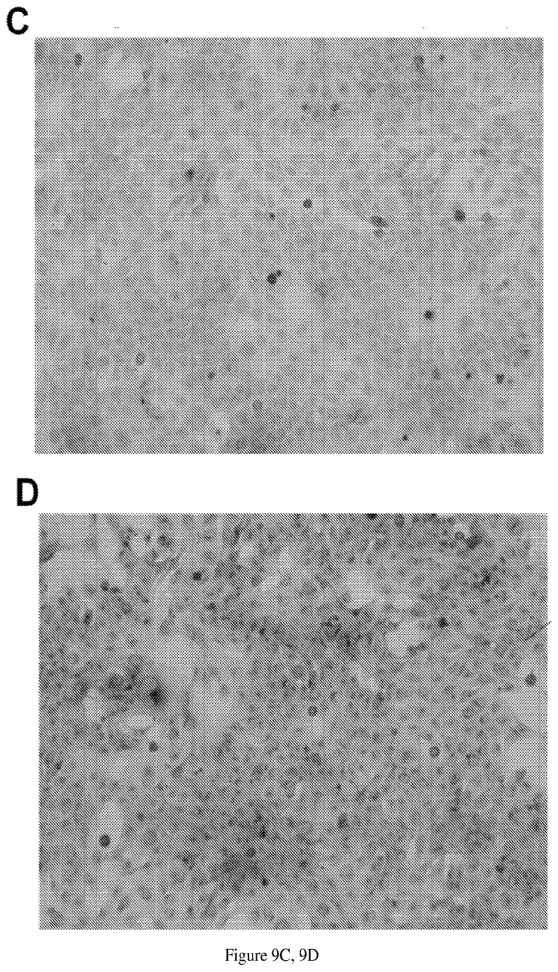

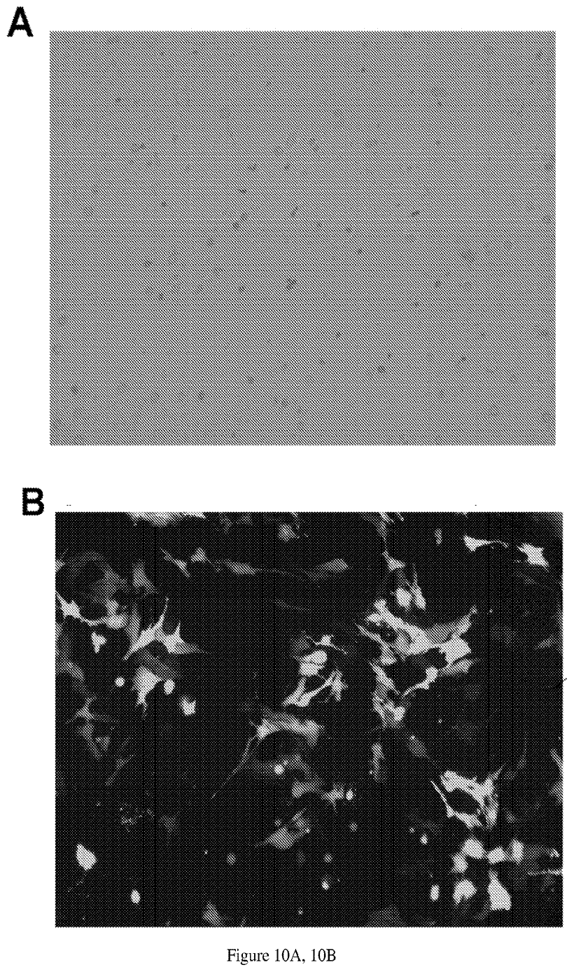

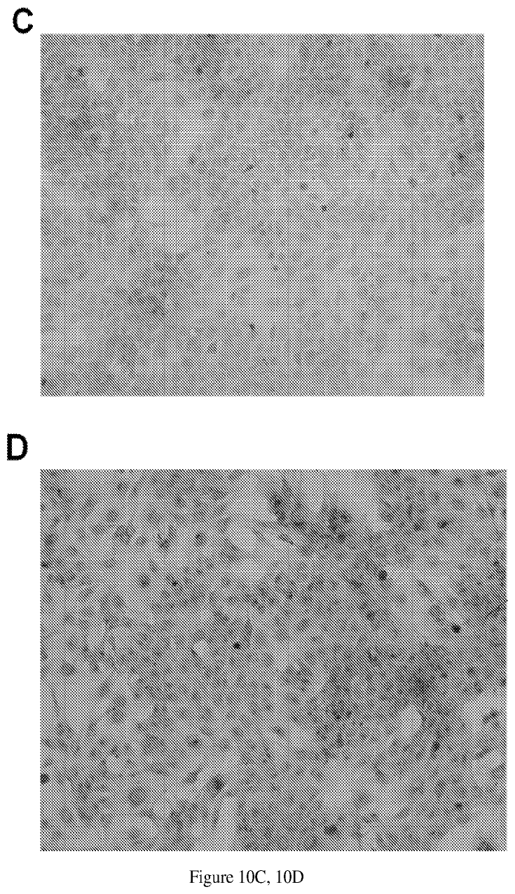

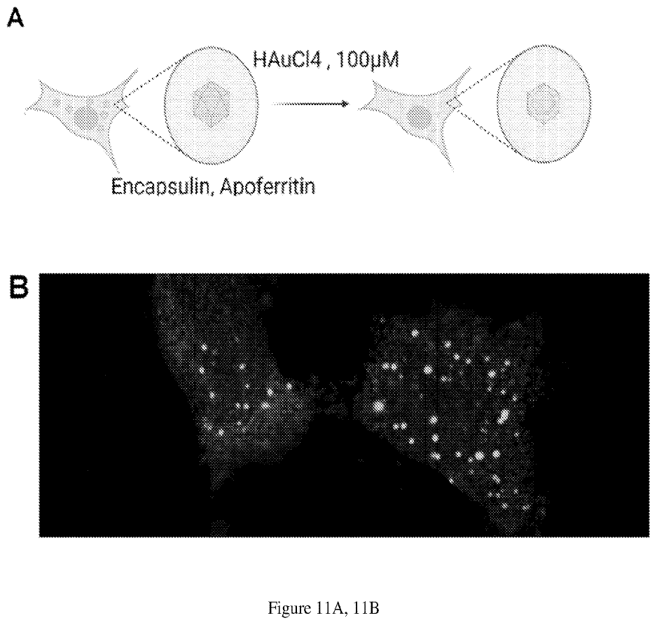

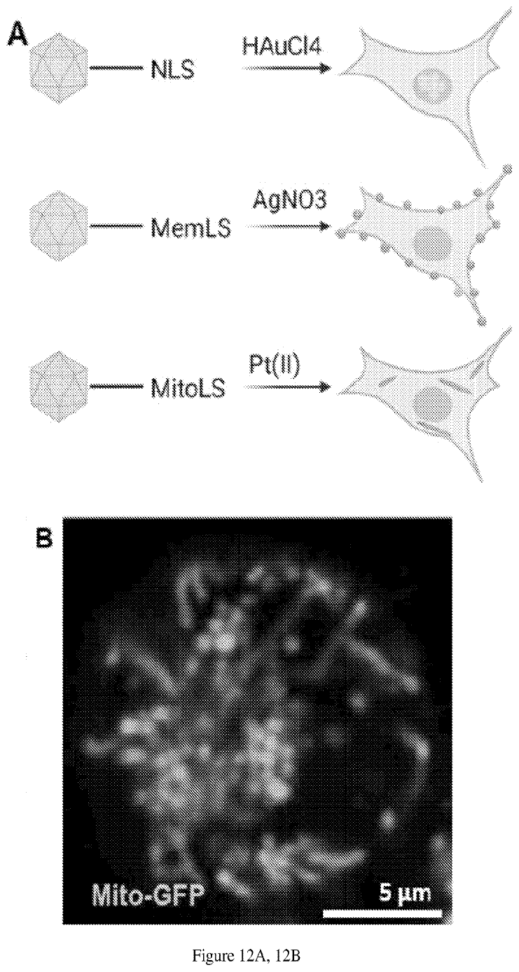

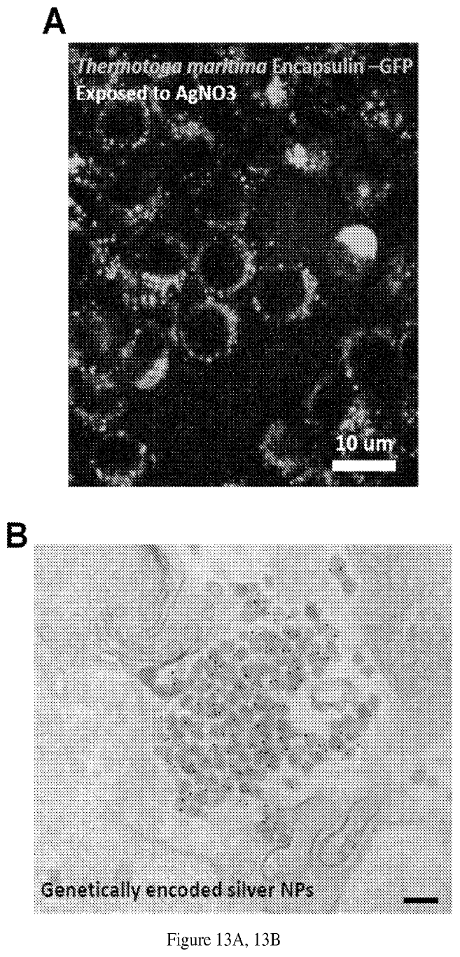

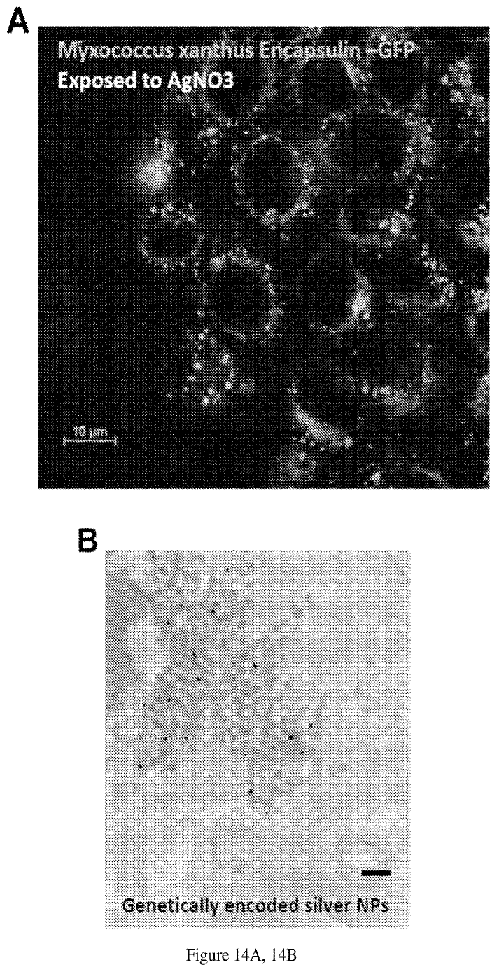

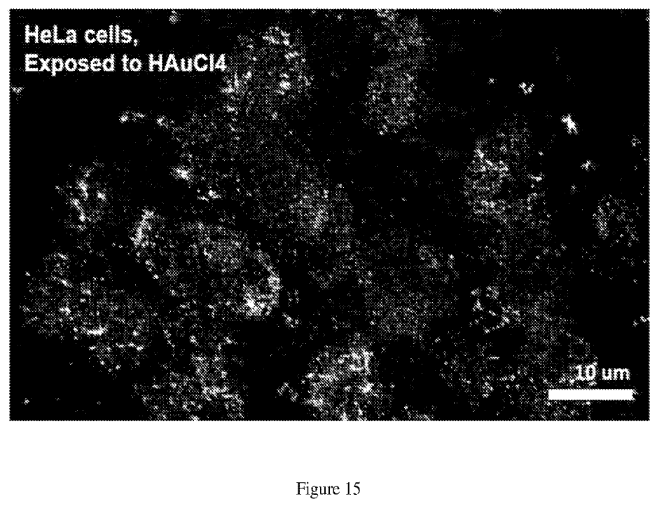

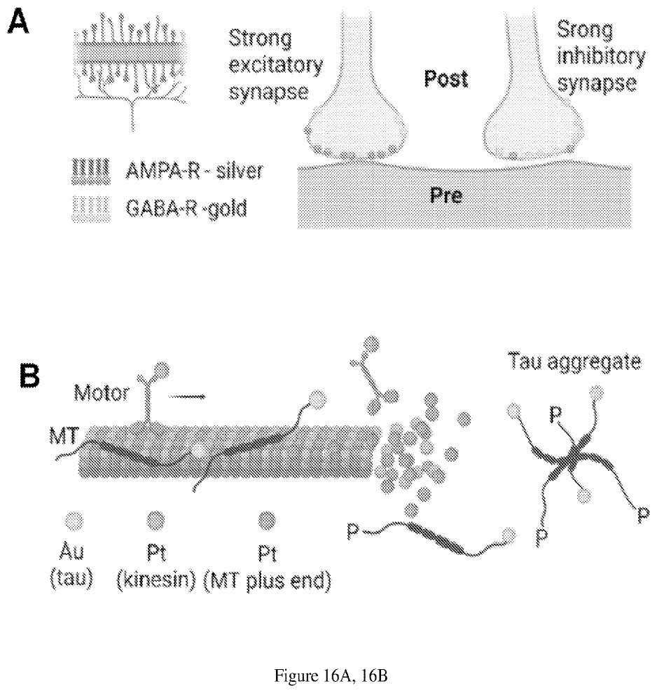

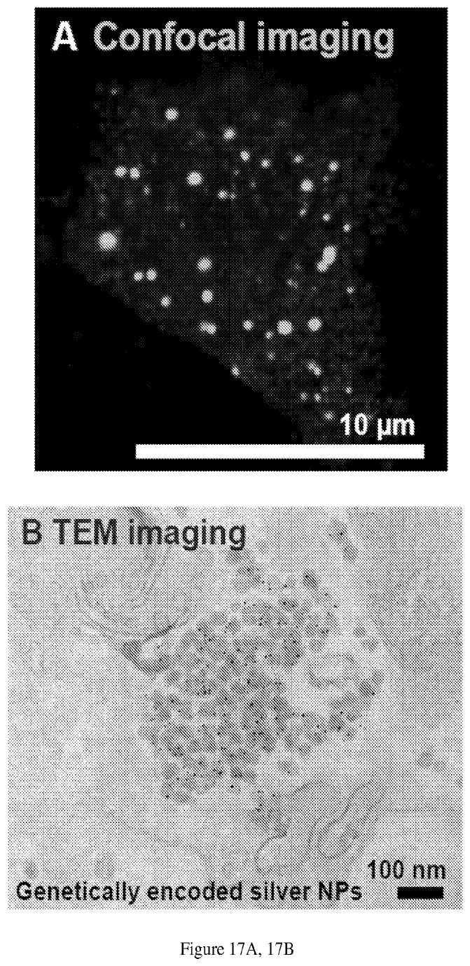

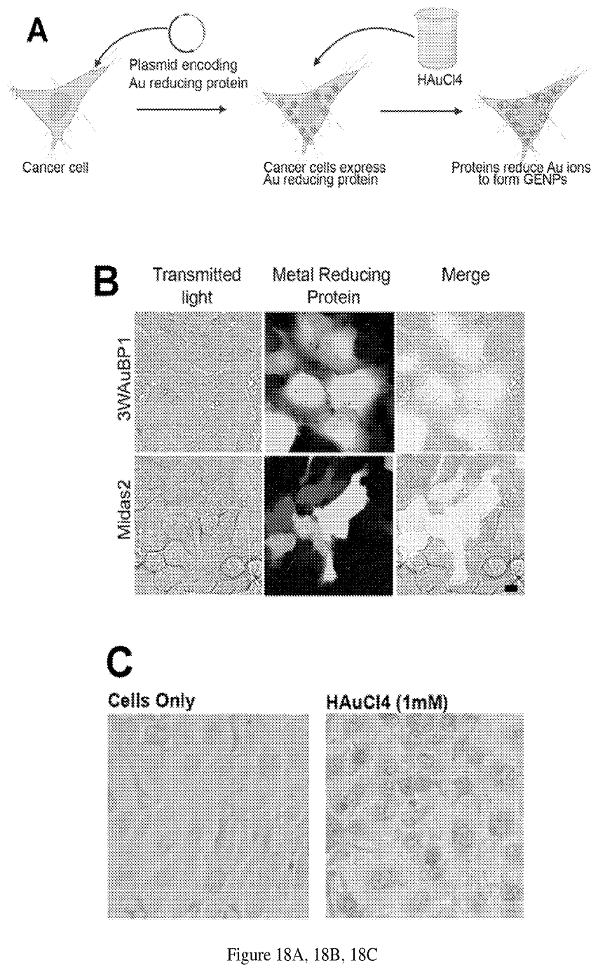

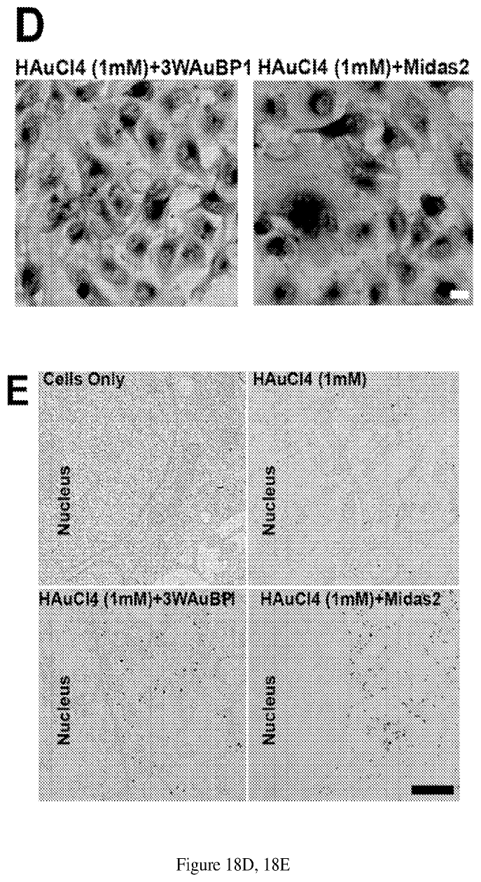

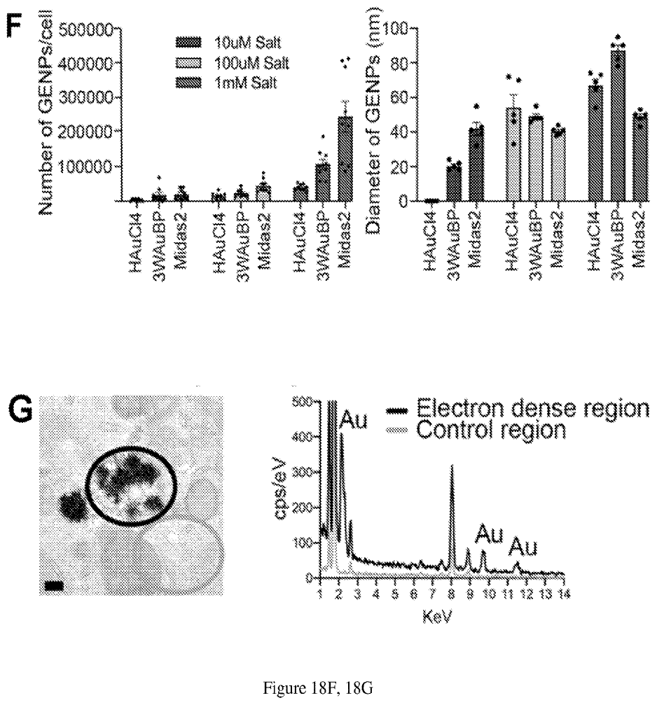

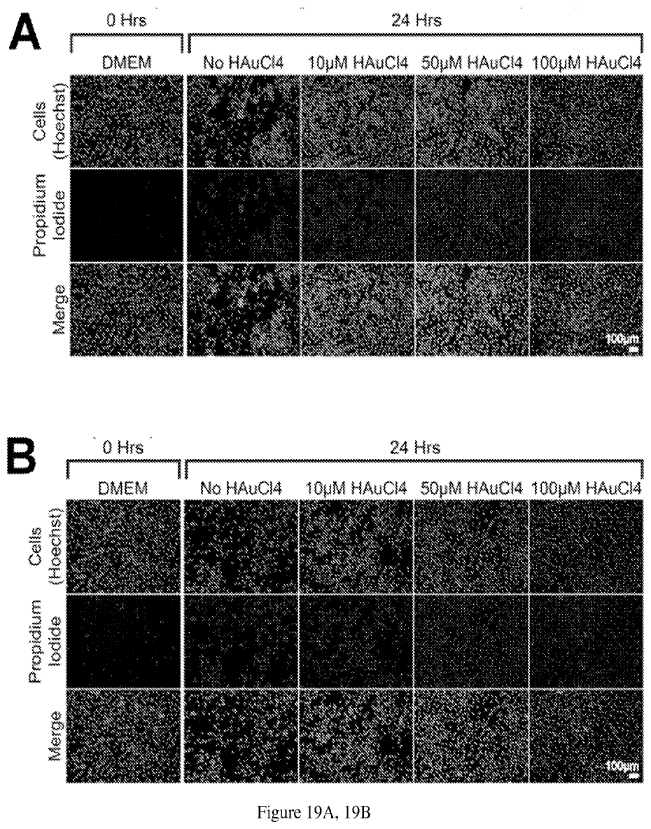

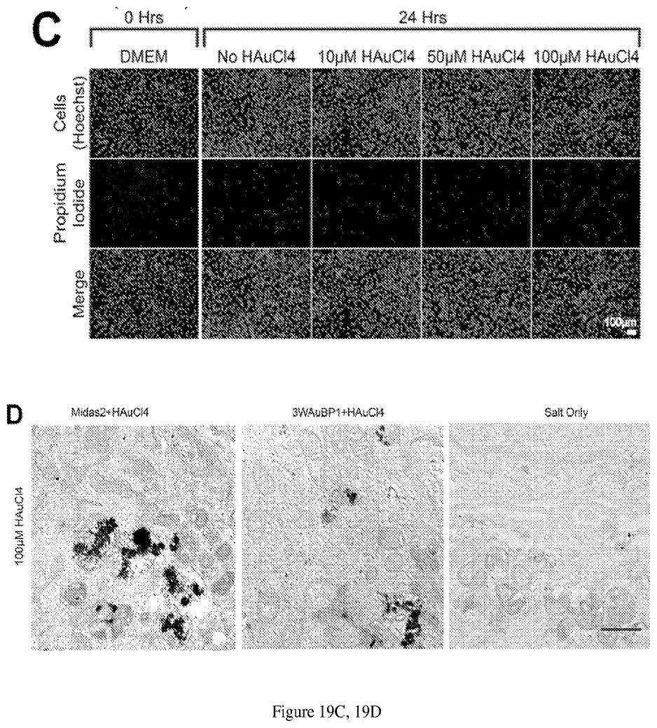

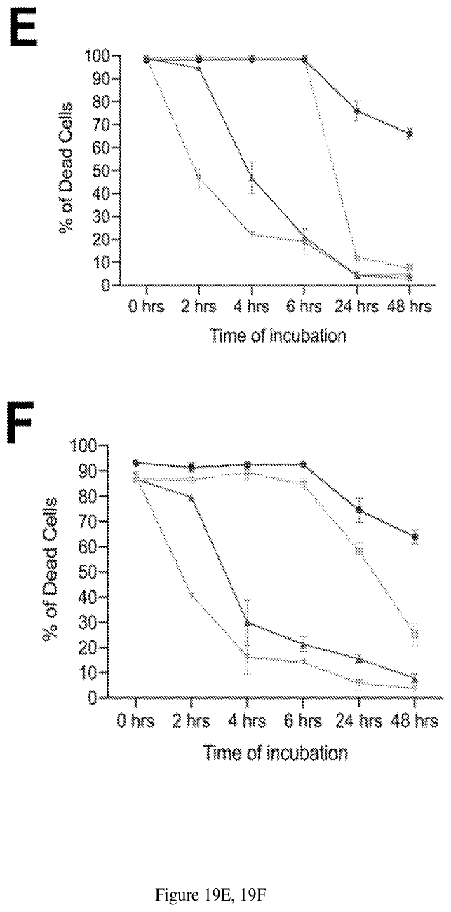

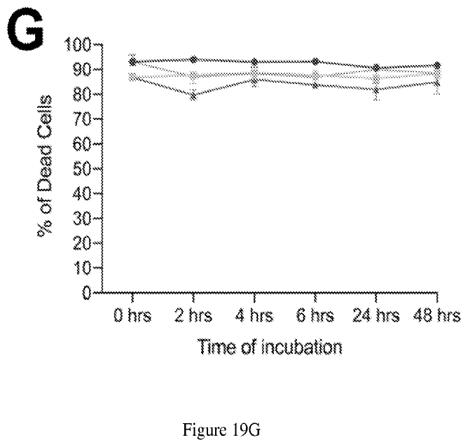

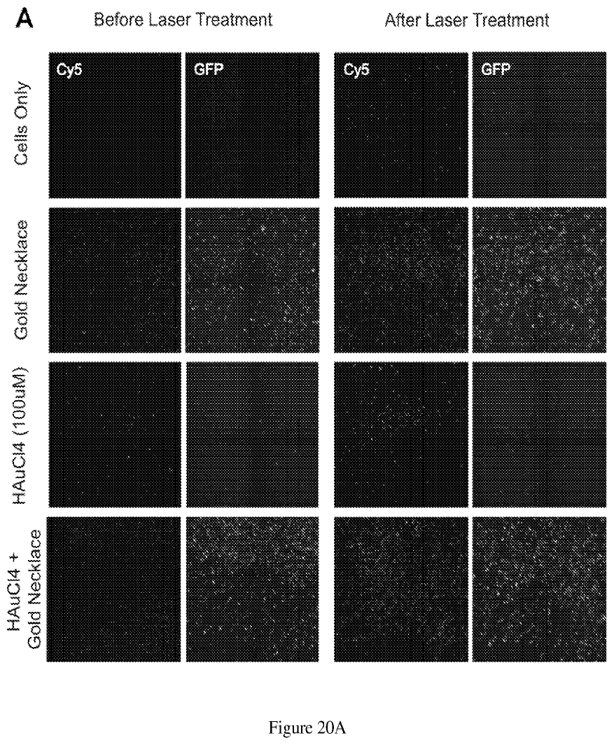

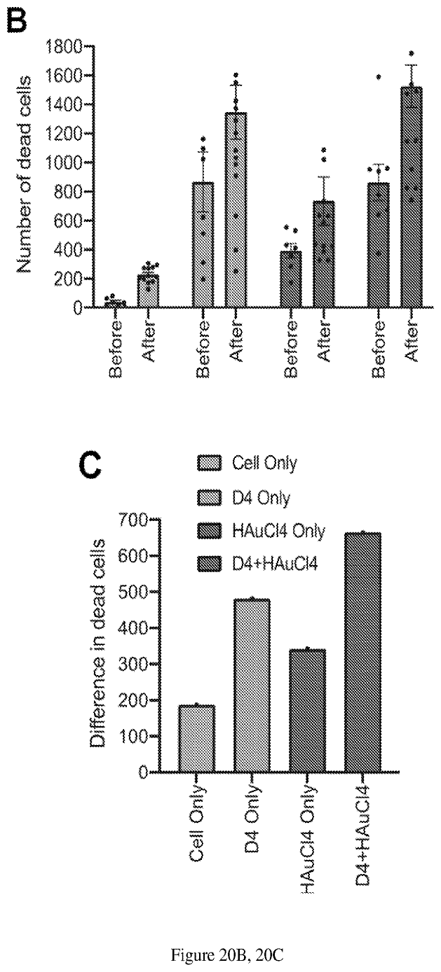

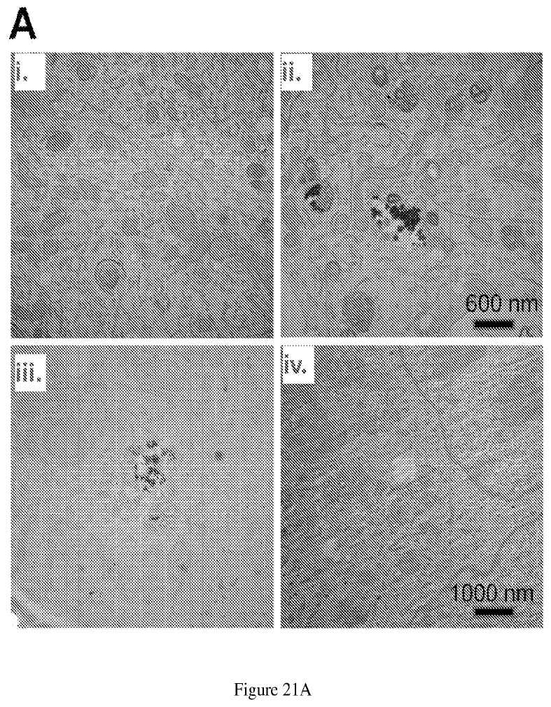

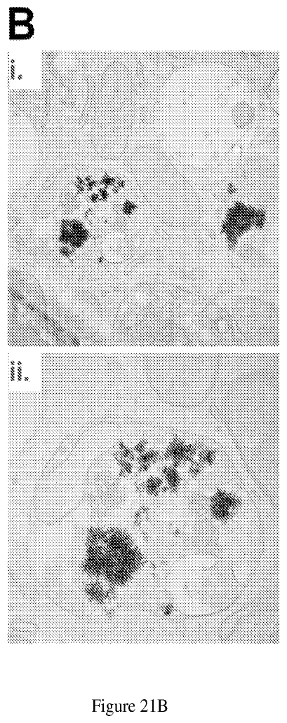

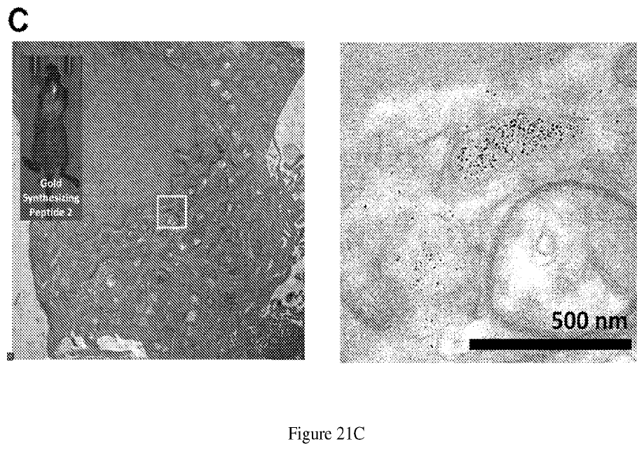

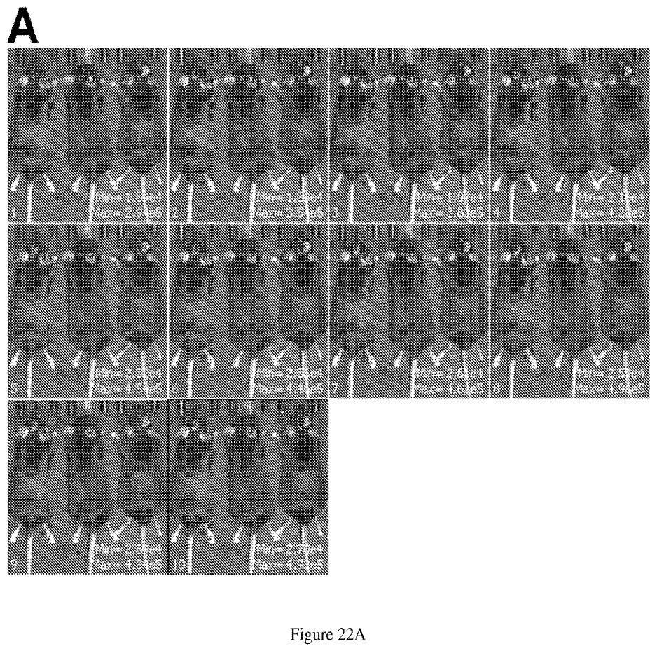

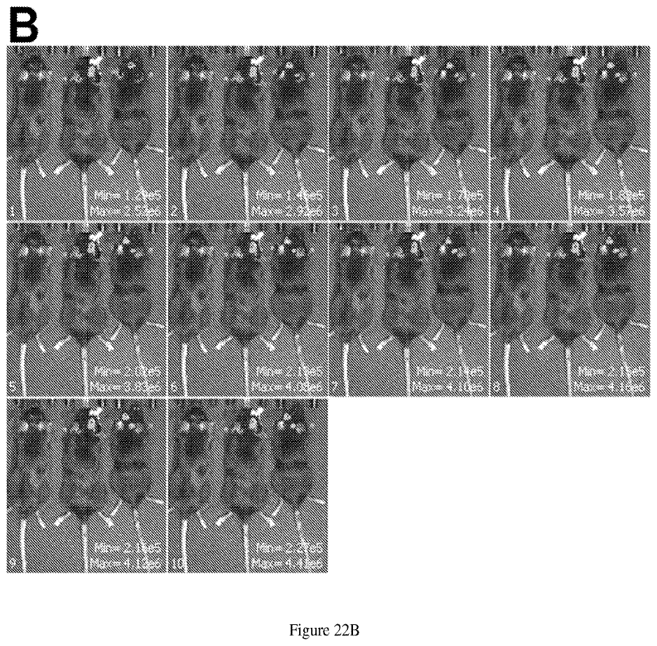

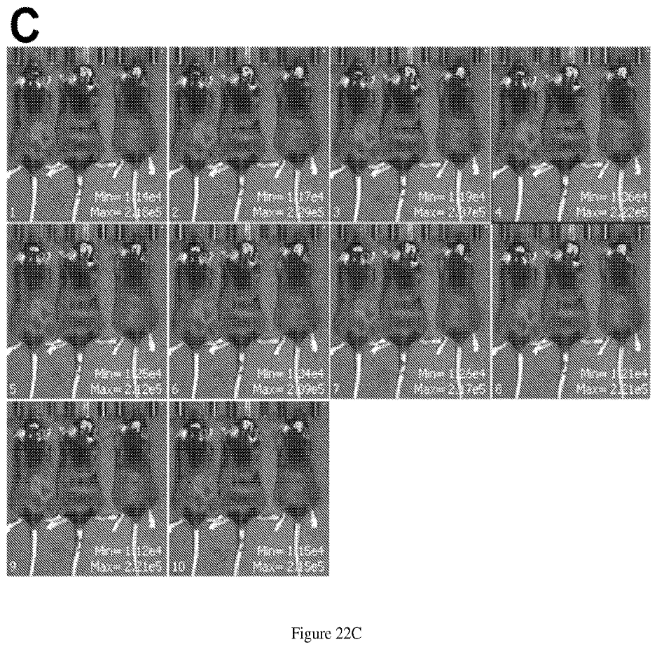

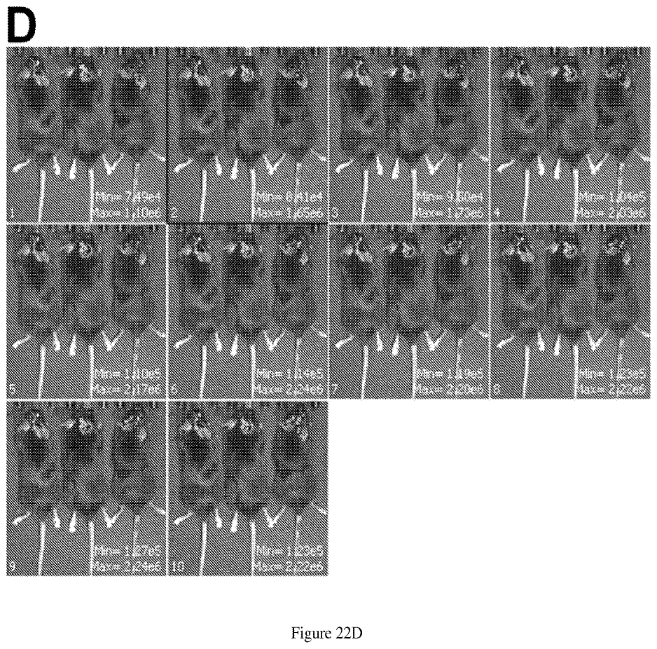

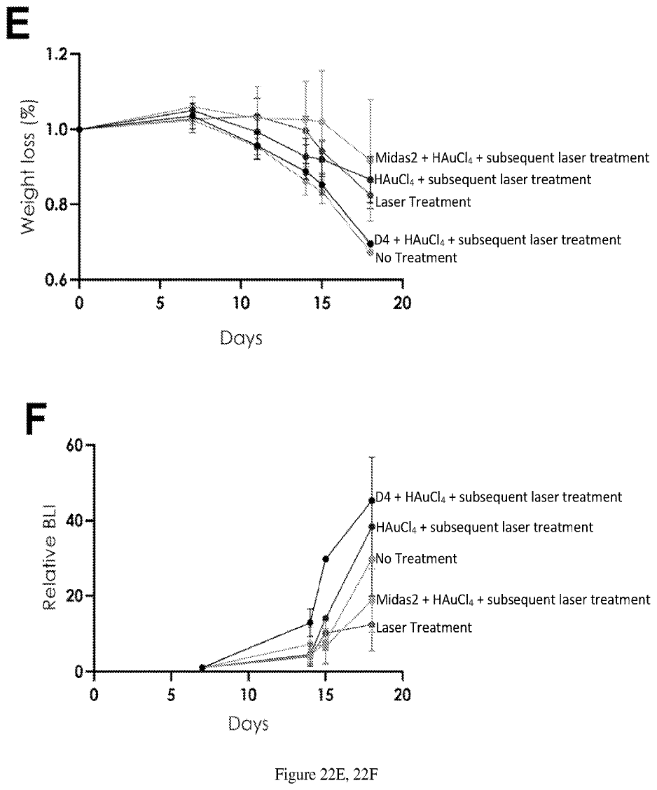

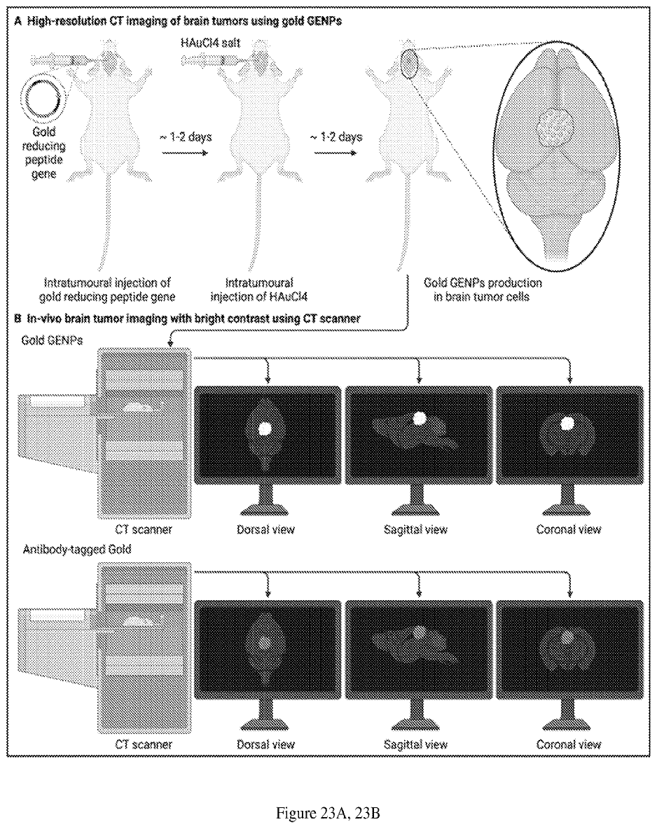

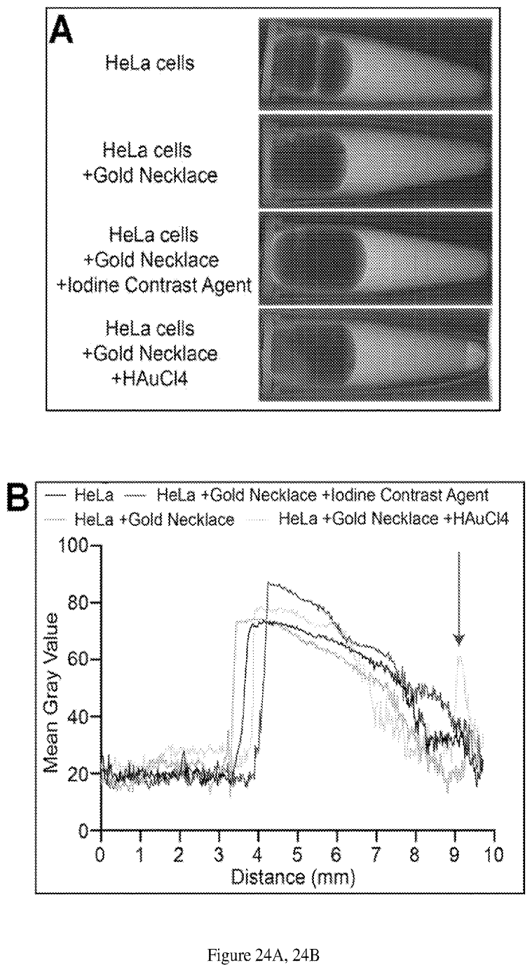

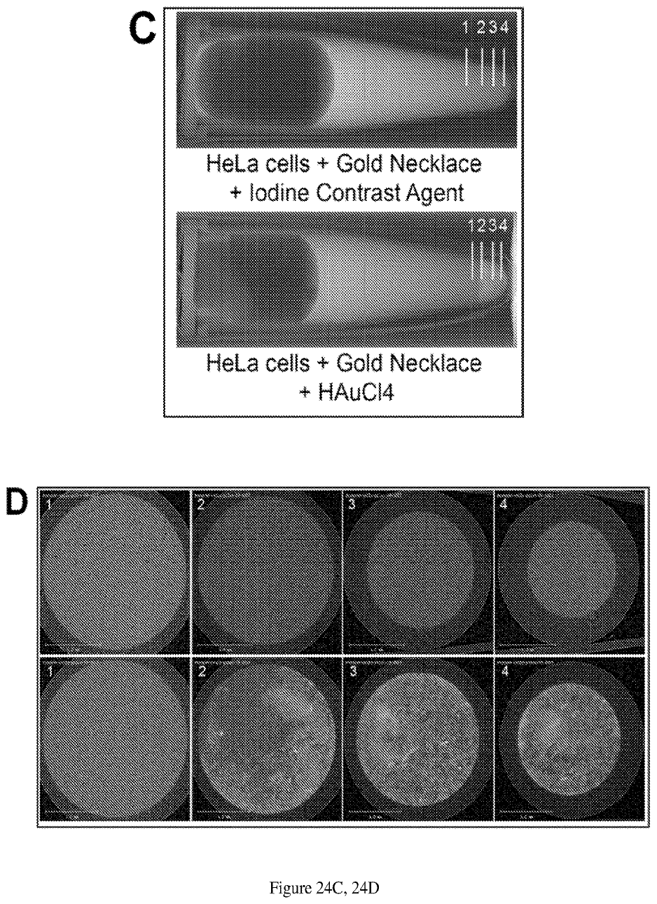

GENP was produced in cancer cells (HeLa) using the standard process. 24 hours following their formation the cells were collected from the wells using a cell scraper and moved to a standard 200 μL PCR tube. Then, the tubes were inserted into a micro-CT (Scanco μCT 50 [Scanco Medical, Brüttisellen, Switzerland] system). Performing scans at 3 μm voxel size, 55 KVp, 145 μA intensity, 0.36 degrees rotation step (180 degrees angular range) and a 1500 ms exposure per view, the PCR tubes containing the GENP bearing cells were imaged. As controls for CT experiments with GENP producing cells, cancers cells that were exposed to salt exclusively were used; cancer cells that express the peptide exclusively; cancer cells that were exposed to iodine-based contrast agent (ISOVUE-300), and finally untreated cancer cells (negative control).

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, the term “agent” refers to a biological substance, such as a chemical, compound, molecule, protein, nucleic acid, or toxin, that can be designed to purposefully fulfill a biological function or action.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (lgs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end.

The term “aptamer” or “aptamers” refers to short, single-stranded DNA or RNA (ssDNA or ssRNA) molecules that can selectively bind to a specific target, including proteins, peptides, carbohydrates, small molecules, toxins, and even live cells. Aptamers are smaller in size compared to antibodies, thus in some embodiments, allow for improved transport and tissue penetration compared to antibodies.

The term “cancer” is used to address any neoplastic disease and is not limited to epithelial neoplasms (surface and glandular cancers; such a squamous cancers or adenomas). It is used here to describe both solid tumors and hematologic malignancies, including epithelial (surface and glandular) cancers, soft tissue and bone sarcomas, angiomas, mesothelioma, melanoma, lymphomas, leukemias and myeloma. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, non-small cell lung cancer (NSCLC), or pancreatic cancer.

As used herein, the terms “neoplastic cells,” “neoplasia,” “tumor,” “tumor cells,” “cancer,” and “cancer cells” (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign. A metastatic cell or tissue means that the cell can invade and destroy neighboring body structures.

The terms “cell,” “cell line” and “cell culture” include progeny. It is also understood that all progenies may not be precisely identical in DNA content, due to deliberate or inadvertent mutations. Variant progeny that have the same function or biological property, as screened for in the originally transformed cell, are included. The “host cells” used in the present invention generally are prokaryotic or eukaryotic hosts.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

A “decrease” or “reduction” can refer to any change that results in a smaller amount of a symptom, disease, composition, condition, or activity. A decrease or reduction can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

As used herein, the term “encapsulate” or “encapsulating” refers to a process in which molecules, such as metal ions, and/or other macromolecules are surrounded or coated partially, almost completely or completely.

As used herein, the term “expression” refers to either or both “gene expression” and “protein expression.” “Gene expression” refers to the process by which polynucleotides are transcribed into mRNA and “protein expression” refers to the process by which mRNA is translated into peptides, polypeptides, or proteins.

A “fluorophore” is a fluorescent chemical compound that can re-emit light upon light excitation. The chemicals are sometimes used alone as a tracer in fluids, as a due for staining certain structures, as an enzyme substrate, or as a probe/indicator. More commonly they are covalently bonded to a macromolecule to serve as a marker for bioactive reagents (ie: antibodies, peptides, nucleic acids, etc.) Fluorophores are notably used to stain tissues, cells, or materials in a variety of analytical methods such as fluorescent imaging and spectroscopy.

A “gene” refers to a polynucleotide containing at least one open reading frame that is capable of encoding a particular polypeptide or protein after being transcribed and translated. Any of the polynucleotides sequences described herein may be used to identify larger fragments or full-length coding sequences of the gene with which they are associated.

As used herein, the term “infection” refers to the entry of tissues by a pathogen, their multiplication, and reaction of host tissues to the pathogen and any toxins they release. Infections can be caused by a wide range of pathogens, most common are bacteria and viruses.

The term “identity” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. Neither N-nor C-terminal extensions nor insertions shall be construed as reducing identity or homology. A polynucleotide or polynucleotide region (or a polypeptide or polypeptide region) that has a certain percentage (for example, 80%, 85%, 90%, or 95%) of “sequence identity” to another sequence means that, when aligned over their full lengths, that percentage of bases (or amino acids) are the same in comparing the two sequences. This alignment and the percent homology or sequence identity can be determined using software programs known in the art. In one embodiment, default parameters are used for alignment. In one embodiment a BLAST program is used with default parameters. In one embodiment, BLAST programs BLASTN and BLASTP are used with the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

As used herein, “operably linked” refers to two or more compositions or compounds being bound or linked together in such a way the optimizes the intended function. When bound or linked, these compositions or compounds can be linked covalently, through electrostatic interaction, through hydrogen bonding, or any combinations thereof.

“Optional” or “optionally” means that the subsequently described event or circumstance may or may not occur, and that the description includes instances where said event or circumstance occurs and instances where it does not.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The terms “prevent,” “preventing,” “prevention,” and grammatical variations thereof as used herein, refer to a method of partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

Reference also is made herein to peptides, polypeptides, proteins and compositions comprising peptides, polypeptides, and proteins. As used herein, a polypeptide and/or protein is defined as a polymer of amino acids, typically of length ≥100 amino acids (Garrett & Grisham, Biochemistry, 2nd edition, 1999, Brooks/Cole, 110). As used herein, The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

The peptides, polypeptides, and/or proteins disclosed herein may comprise the entire length of a defined amino acid sequence or may a shorter length of a longer amino acid sequence. For example, the length of a fragment sequence is taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination. In some embodiments, the term “therapeutically effective” refers to the amount of a compound such as a metal ion or a nanoparticle containing a metal ion that will elicit the biological or medical response of a tissue, system, animal, or human that is being sought by the researcher, veterinarian, medical doctor or other clinician over a generalized period of time. In some embodiments, a desired response is a reduction in a cancer, an infection or radiation damage. In some instances, a desired biological or medical response is achieved following administration of multiple dosages of the composition to the subject over a period of days, weeks, or years.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. In some embodiments the subject is devoid of disease or healthy. In some embodiments, the subject has cancer. In some embodiments the subject has an infection.

The terms “therapeutically effective amount” or “therapeutically effective dose” refer to the amount of a compound such as an anti-cancer composition that will elicit the biological or medical response of a tissue, system, animal, or human that is being sought by the researcher, veterinarian, medical doctor or other clinician over a generalized period of time. In some embodiments, a desired response is improvement in diseases like cancer. In some instances, a desired biological or medical response is achieved following administration of multiple dosages of the composition to the subject over a period of days, weeks, or years.

The terms “treat,” “treating,” “treatment,” and grammatical variations thereof as used herein, include partially or completely alleviating, mitigating, or reducing the intensity of one or more attendant symptoms of a disorder or condition and/or alleviating or mitigating one or more causes of a disorder or condition. Treatments according to the disclosure may be applied palliatively or remedially. Treatments are administered during early onset (e.g., upon initial signs and symptoms of cancer) or after an established development of the disorder or condition (e.g., cancer).

In some instances, the terms “treat”, “treating”, “treatment” and grammatical variations thereof, include partially or completely reducing the size of a tumor, reducing the number of tumors, and reducing the severity of a cancer as compared with prior to treatment of the subject or as compared with the incidence of such symptom in a general or study population. The terms “treat”, “treating”, “treatment” and grammatical variations thereof, can also include decreasing tumor resistance as compared with prior to treatment of the subject or as compared with the incidence of such symptom in a general or study population. The terms “treat”, “treating”, “treatment” and grammatical variations thereof, can also include decreasing a microbial infection as compared with prior to treatment of the subject or as compared with the incidence of such symptom in a general or study population. The terms “treat”, “treating”, “treatment” and grammatical variations thereof, can also include partially or completely reducing an inflammatory response from a cancer or microbial infection as compared with prior to treatment of the subject or as compared with the incidence of such symptom in a general or study population.

The word “vector” refers to any vehicle that carries a polynucleotide into a cell for the expression of the polynucleotide in the cell. The vector may be, for example, a plasmid, a virus, a phage particle, or a nanoparticle. Once transformed into a suitable host, the vector may replicate and function independently of the host genome, or may in some instances, integrate into the genome itself. In some embodiments, the vector is a DNA construct containing a DNA sequence which is operably linked to a suitable control sequence capable of affecting the expression of the DNA in a suitable host cell. Such control sequences can include a promoter to effect transcription, an optional operator sequence to control such transcription, a sequence encoding suitable mRNA ribosome binding sites, and sequences which control the termination of transcription and translation. In other embodiments, the vector is a lipid nanoparticle. Lipid nanoparticles can be used to deliver mRNA to a host cell for expression of the mRNA in the host cell.

### Methods of Making Nanoparticle Compositions

The present disclosure provides methods of making nanoparticle compositions. In some embodiments, the nanoparticle is formed within a cell (intracellularly). In some aspects, disclosed herein is a method of making a nanoparticle comprising one or more metals in a cell, the method comprising expressing one or more nucleic acid sequences encoding one or more metal binding peptides and one or more metal reducing peptides in the cell, administering a composition comprising an ion of each of the one or more metals, or a salt thereof, to the cell, and forming the nanoparticle within the cell upon reduction of the ion of each of the one or more metals. In some embodiments, a peptide has both metal binding and metal reducing capabilities and is referred to herein as a “combined metal binding and metal reducing peptide.”

Accordingly, in some embodiments, the nanoparticle comprises a metal. In some embodiments, the nanoparticle comprises one or more metals selected from the group consisting of gold (Au), silver (Ag), platinum (Pt), copper (Cu), palladium (Pd), rhenium (Re), zinc (Zn), ruthenium (Ru), cobalt (Co), cadmium (Cd), aluminum (Al), nickel (Ni), and iron (Fe). In some embodiments, the one or more metals are selected from the group consisting of gold, silver, zinc, titanium, lead and potassium. In some embodiments, one of the one or more metals comprises gold. In some embodiments, one of the one or more metals comprises silver. In some embodiments, one of the one or more metals comprises zinc. In some embodiments, one of the one or more metals comprises titanium. In some embodiments, one of the one or more metals comprises lead. In some embodiments, one of the one or more metals comprises potassium. In some embodiments, the one metal is gold. In some embodiments, the one metal is silver. In some embodiments, one of the one or more metals is gold. In some embodiments, the metals are gold and silver.

In some embodiments, one of the one or more metals is gold and the composition comprising the ion is chloroauric acid (HAuCl₄), or any salt thereof. In some embodiments, one of the one or more metals is silver and the composition comprising the ion is silver nitrate (AgNO₃), or any salt thereof. In some embodiments, one of the one or more metals is zinc and the composition comprising the ion is zinc oxide (ZnO), or any salt thereof. In some embodiments, one of the one or more metals is titanium and the composition comprising the ion is titanium dioxide (TiO₂), or any salt thereof. In some embodiments, one of the one or more metals is lead and the composition comprising the ion is lead sulfide (PbS), or any salt thereof. In some embodiments, one of the one or more metals is potassium and the composition comprising the ion is potassium tetrachloroplatinate (K₂PtCl₄), or any salt thereof.

Also included herein are methods wherein the metal ion is administered to the cell in a subtoxic amount. As used herein, “subtoxic amount” refers to an amount that allows for expression of the nucleic acid sequence within the cell and formation of the nanoparticle. In some embodiments, a “subtoxic amount” is an amount that does not kill the cell.

As described herein, the one or more metal binding peptides encoded by the nucleic acid bind to the one or more metals comprised within the nanoparticle. For example, when the nanoparticle comprises gold, the metal binding peptide and/or the combined metal binding and metal reducing peptide binds gold. In other or further embodiments, the nanoparticles comprises silver and the metal binding peptide and/or the combined metal binding and metal reducing peptide binds silver. In some embodiments, the nucleic acid sequence encodes one, two, three, four, five, six, seven, eight, nine, ten, eleven, twelve or more same or different metal binding peptides and/or combined metal binding and reducing peptides. In some embodiments, the nucleic acid sequence encodes one, two, three, four, five, six, seven, eight, nine, ten, eleven, twelve or more same or different gold binding peptides and/or combined gold binding and reducing peptides. In some embodiments, the nucleic acid sequence encodes four to eight gold binding peptides and/or combined gold binding and reducing peptides, wherein the gold binding peptides are the same or different. In some embodiments, the nucleic acid sequence encodes five to seven gold binding peptides and/or combined gold binding and reducing peptides, wherein the gold binding peptides are the same or different. In some embodiments, the multiple same or different gold binding peptides and/or combined gold binding and reducing peptides are separated by linker peptides. In some embodiments, the nucleic acid sequence encodes six gold binding peptides and/or combined gold binding and reducing peptides, wherein the gold binding peptides are the same or different. In some embodiments, the nucleic acid sequence encodes a peptide comprising SEQ ID NO: 28 (referred to herein as D4 or GOLD NECKLACE). Using multiple repeats of nucleic acids encoding metal binding peptides and/or combined metal binding and reducing peptides can increase the metal ion reducing capability.

In some embodiments, the one or more gold metal binding peptides comprises a sequence SEQ ID NO: 1. In some embodiments, the one or more gold metal binding peptides have at least 70% sequence identity to SEQ ID NO: 1. In some embodiments, the one or more gold metal binding peptides have 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 1. In some embodiments, the one of more gold metal binding peptides comprise SEQ ID NO: 1.

In some embodiments, the nucleic acid sequence comprises a sequence that encodes a combined metal binding peptide and metal reducing peptide. For example, SEQ ID NO:2, also referred to herein as “Midas2,” can function as both a gold binding peptide and a gold reducing peptide. Accordingly, in some embodiments, the one or more combined gold metal binding peptide and the gold reducing peptide have at least 70% sequence identity to SEQ ID NO: 2. In some embodiments, the one or more combined gold metal binding peptide and the gold reducing peptide have 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2. In some embodiments, the one or more combined gold metal binding peptide and the gold reducing peptide comprises SEQ ID NO: 2.

In some embodiments, the one or more combined gold metal binding peptide and the gold reducing peptide have at least 70% sequence identity to SEQ ID NO: 28. In some embodiments, the one or more combined gold metal binding peptide and the gold reducing peptide have 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 28. In some embodiments, the one or more combined gold metal binding peptide and the gold reducing peptide comprises SEQ ID NO: 28.

In some embodiments, one of the one or more metals is silver and the composition comprising the ion is silver nitrate (AgNO₃). In some embodiments, the one or more silver metal binding peptides have at least 70% sequence identity to SEQ ID NO: 7. In some embodiments, the one or more silver metal binding peptides have 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 7. In some embodiments, the one or more silver metal binding peptides comprise SEQ ID NO: 7.

In some embodiments, the one or more silver metal binding peptides have at least 70% sequence identity to SEQ ID NO: 27. In some embodiments, the one or more silver metal binding peptides have 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 27. In some embodiments, the one or more silver metal binding peptides comprise SEQ ID NO: 27.

In some embodiments, the nucleic acid sequences encodes one, two, three, four, five, seven, eight, nine, ten, eleven, twelve or more same or different silver binding peptides. In some embodiments, the nucleic acid sequence encodes four to eight silver binding peptides, wherein the silver binding peptides are the same or different. In some embodiments, the nucleic acid sequence encodes five to seven silver binding peptides, wherein the silver binding peptides are the same or different. In some embodiments, the nucleic acid sequence encodes six silver binding peptides, wherein the silver binding peptides are the same or different. In some embodiments, the multiple same or different silver binding peptides are separated by linker peptides.

Each of Tryptophan (Trp or W) and histidine (His or H) can reduce metal ions. Accordingly, in some embodiments, the metal reducing peptide comprises tryptophan. In other or further embodiments, the metal reducing peptide comprises histidine. In some embodiments, the metal reducing peptide comprises or consists of 1, 2, 3, 4, 5, 6, 7, 8, 9, 10, 11, 12, 13, 14, 15, or more tryptophan amino acids. In some embodiments, the metal reducing peptide comprises or consists of 1, 2, 3, 4, 5, 6, 7, 8, 9, 10, 11, 12, 13, 14, 15, or more histidine amino acids. In some embodiments, the nucleic acid sequence encodes any combination of 1, 2, 3, 4, 5, 6, 7, 8, 9, 10, 11, 12, 13, 14, 15, or more tryptophan and histidine amino acids.

In some embodiments, the one or more metal reducing peptide comprises at least one tryptophan amino acid. In some embodiments, the one or more metal reducing peptide comprises 1, 2, 3, 4, 5, 6, 7, 8, 9, 10, 11, 12, 13, 14, 15, or more tryptophan amino acids. In some embodiments, the one or more gold reducing peptide sequence comprises or consists of WW. In some embodiments, the one or more gold reducing peptide sequence comprises or consists of WWW. In some embodiments, the one or more gold reducing peptide sequence comprises or consists of WWWW (SEQ ID NO: 3). In some embodiments, the one or more gold metal reducing peptide sequence comprises or consists of WWWWW (SEQ ID NO: 4). In some embodiments, the one or more gold metal reducing peptide sequence comprises or consists of WWWWWW (SEQ ID NO: 5). In some embodiments, the one or more gold metal reducing peptide sequence comprises or consists of WWWWWWW (SEQ ID NO: 6). In some embodiments, the metal reducing peptide comprises at least one histidine amino acid. In some embodiments, the metal reducing peptide comprises 1, 2, 3, 4, 5, 6, 7, 8, 9, 10, 11, 12, 13, 14, 15, or more histidine amino acids.

In some embodiments, the nucleic acid sequence further encodes one or more linkers. In some embodiments, the nucleic acid sequence comprises 1, 2, 3, 4, 5, 6, 7, 8, 9, 10, 11, 12, 13, 14, 15, 16, 17, 18, 19, 20, or more linkers. In some embodiments, the linker comprises SEQ ID NO:8 (GGGGSGGGGS), or a fragment thereof. In some embodiments, the linker comprises GSG. In some embodiments, the nucleic acid sequence encodes 1, 2, 3, 4, 5, 6, 7, 8, 9, 10, 11, 12, 13, 14, 15, 16, 17, 18, 19, 20, or more linkers comprising SEQ ID NO:8. In some embodiments, the nucleic acid sequence encodes one linker, In some embodiments, the nucleic acid sequence encodes two linkers. In some embodiments, the nucleic acid sequence encodes a sequence wherein a linker is placed between each successive metal binding and reducing peptide. In some embodiments, the nucleic acid sequence encodes a fragment of SEQ ID NO: 8. In some embodiments, the nucleic acid sequence encodes at least 50% of SEQ ID NO: 8.

Herein, the nucleic acid sequence can encode any combination of metal binding peptides of any preceding aspect, metal reducing peptides of any preceding aspect, and linker of any preceding aspect needed to produce the desired effect. Non-limiting examples includes a nucleic acid sequence encoding a WWW-linker-AuBP1, AuBP1-linker-WWW, WWW-linker-Midas2, or Midas2-linker-WWW. Additional examples are provided in Example 4 below.

In some embodiments, an expression vector comprises the nucleic acid sequence. In some embodiments, the expression vector comprises a plasmid or a virus or viral vector. A plasmid or a viral vector can be capable of extrachromosomal replication or, optionally, can integrate into the host genome. As used herein, the term “integrated” used in reference to an expression vector (e.g., a plasmid or viral vector) means the expression vector, or a portion thereof, is incorporated (physically inserted or ligated) into the chromosomal DNA of a host cell. As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). Suitable viruses and viral vectors include adenovirus, lentivirus, retrovirus, among others. In some embodiments, the expression vector further comprises a suitable control sequence capable of effecting the expression of the DNA in a suitable host cell. Such control sequences can include a promoter to effect transcription, an optional operator sequence to control such transcription, a sequence encoding suitable mRNA ribosome binding sites, and sequences which control the termination of transcription and translation.

In some embodiments, the nucleic acid sequence further comprises a sequence encoding one or more cage-like peptides, or a fragment thereof. It is understood that the term “cage-like peptide” refers to a peptide that forms a closed or nearly closed encapsulating structure either alone or when in association or contact with other peptides or molecules. In some embodiments, more than one cage-like peptide encapsulates or surrounds a metal ion to form a nanoparticle. In some embodiments, the nucleic acid sequence encodes 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, or more cage-like peptides.

In some embodiments, the one or more cage-like peptides are selected from the group consisting of apoferritin, encapsulin, sericin, laccase, and ligninase. In some embodiments, the one cage-like peptide is an encapsulin peptide, and the nucleic acid sequence encodes one or more fragments of the encapsulin peptide. In some embodiments, the nucleic acid sequence encodes one or more of SEQ ID NO:20, SEQ ID NO:21, SEQ ID NO:23, and SEQ ID NO:24.

Encapsulins are a family of bacterial proteins that can serve as the main structural components of encapsulin-based nanoparticles. In some embodiments, the encapsulin protein comprises EncA. In some embodiments, the encapsuling protein comprises EncB. In some embodiments, the encapsulin protein comprises EncC. In some embodiments, the encapsulin protein comprises EncD. In some embodiments, Encapsulin proteins self-assemble to form icosahedral structures of various diameters (24-42 nm). In some embodiments, the self-assembled encapsulin icosahedral forms nanocompartment shells. In some embodiments, the nanocompartment can be loaded with various compounds, such as drugs, which makes it possible to create targeted drug delivery systems. In some embodiments, the nanocompartment shell can be labeled for optical and MRI imaging.

### Methods of Using Nanoparticle Compositions

Also included herein are methods of using the nanoparticle compositions prepared according to the methods described herein. In some embodiments, the nanoparticle compositions are used for cancer treatment. In these embodiments, the nanoparticle is formed in a cancer cell in a subject according to a method described herein, and a therapeutically effective amount of an electromagnetic radiation is administered to the cell. In some aspects, the nanoparticle comprises a gold nanoparticle, a silver nanoparticle, a platinum nanoparticle, copper nanoparticle, a palladium nanoparticle, rhenium nanoparticle, a zinc nanoparticle, a ruthenium nanoparticle, a cobalt nanoparticle, a cadium nanoparticle, an aluminum nanoparticle, a nickel nanoparticle, or an iron nanoparticle. As used herein, a therapeutically effective amount of an electromagnetic radiation includes an amount that kills the cell, and a treatment of the cancer includes a reduction in the size of the tumor.

In some embodiments, the cancer includes, but is not limited to acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva). In some embodiments, the cancer is a brain cancer.

In other embodiments, the nanoparticle compositions are used for treatment of a microbial infection. In these embodiments, the microbial infection includes, but is not limited to a bacterial infection, a viral infection, a fungal infection, a parasitic infection, and combinations thereof. In some embodiments, the microbial infection includes, but is not limited to common cold, influenza (including, but not limited to human, bovine, avian, porcine, and simian strains of influenza), measles, acquired immune deficiency syndrome/human immunodeficiency virus (AIDS/HIV), anthrax, botulism, cholera, campylobacter infections, chickenpox, chlamydia infections, cryptosporidiosis, dengue fever, diphtheria, hemorrhagic fevers, Escherichia coli (E. coli) infections, ehrlichiosis, gonorrhea, hand-foot-mouth disease, hepatitis A, hepatitis B, hepatitis C, legionellosis, leprosy, leptospirosis, listeriosis, malaria, meningitis, meningococcal disease, mumps, pertussis, polio, pneumococcal disease, paralytic shellfish poisoning, rabies, rocky mountain spotted fever, rubella, salmonella, shigellosis, small pox, syphilis, tetanus, trichinosis (trichinellosis), tuberculosis (TB), typhoid fever, typhus, west nile virus, yellow fever, yersiniosis, or zika.

In some embodiments, the microbial infection is derived from a bacteria, virus, fungi, archaea, protozoa, algae, protists, including, but not limited to Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, Human Immunodeficiency virus type-2, Mycobacterium tuberculosis (M. tuberculosis), Mycobacterium bovis (M. bovis), Mycobacterium avium (M. avium), Mycobacterium intracellular (M. intracellular), Mycobacterium africanum (M. africanum), Mycobacterium kansasii (M. kansasii), Mycobacterium marinum (M. marinum), Mycobacterium ulcerans (M. ulcerans), Mycobacterium avium (M. avium) subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetii, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neisseria meningitidis, Neisseria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, other Yersinia species, Candida albicans, Cryptococcus neoformans, Histoplama capsulatum, Aspergillus fumigatus, Coccidiodes immitis, Paracoccidioides brasiliensis, Blastomyces dermitidis, Pneumocystis carnii, Penicillium marneffi, Alternaria alternata, Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, and Entamoeba histolytica.

Further included herein are methods of reducing or preventing radiation damage in a cell comprising expressing a nucleic acid sequence encoding one or more metal binding peptides and one or more metal reducing peptides in the cell, administering a composition comprising an ion of each of the one or more metals, or a salt thereof, to the cell, forming the nanoparticle within the cell upon reduction of the ion of each of the one or more metals, and exposing the cell to radiation. In some embodiments, the radiation is x-rays, gamma rays, electron beams, protons, or combinations thereof.

In some embodiments, the cell is in a subject and the method further comprises detecting the location of the nanoparticle in the subject using an imaging method selected from computed tomography (CT), positron emission tomography (PET), magnetic resonance imaging (MRI), and variations thereof.

### Nanoparticle Compositions

In one aspect, disclosed herein is a nanoparticle made by the method of any preceding aspect.

In another aspect, disclosed herein is a nanoparticle composition comprising a metal, one or more metal binding peptides and one or more metal reducing peptides. In some embodiments, the composition comprises the one or more metals selected from the group consisting of gold, silver, zinc, titanium, lead and potassium. In some embodiments, an ion of each of the one or more metals is selected from a group consisting of chloroauric acid (HAuCl₄), silver nitrate (AgNO₃), zinc oxide (ZnO), titanium dioxide (TiO₂), lead sulfide (PbS), potassium tetrachloroplatinate (K₂PtCl₄), and any salt thereof. In some embodiments, the nanoparticle or nanoparticle composition comprises the metal and/or metal ion of any preceding aspect.

In some embodiments, the nanoparticle or nanoparticle composition comprises one, two, three, four, five, six, seven, eight, nine, ten, eleven, twelve or more same or different metal binding peptides and/or combined metal binding and reducing peptides. In some embodiments, the nanoparticle or nanoparticle composition comprises one, two, three, four, five, six, seven, eight, nine, ten, eleven, twelve or more same or different gold or silver binding peptides and/or combined gold or silver binding and reducing peptides. In some embodiments, the nanoparticle or nanoparticle composition comprises four to eight gold or silver binding peptides and/or combined gold or silver binding and reducing peptides, wherein the gold or silver binding peptides are the same or different. In some embodiments, the nanoparticle or nanoparticle composition comprises five to seven gold or silver binding peptides and/or combined gold or silver binding and reducing peptides, wherein the gold or silver binding peptides are the same or different. In some embodiments, the multiple same or different gold or silver binding peptides and/or combined gold or silver binding and reducing peptides are separated by linker peptides. In some embodiments, the nanoparticle or nanoparticle composition comprises six gold or silver binding peptides and/or combined gold or silver binding and reducing peptides, wherein the gold or silver binding peptides are the same or different. In some embodiments, the nanoparticle or nanoparticle composition comprises a peptide comprising SEQ ID NO: 28

In some embodiments, the nanoparticle or nanoparticle composition comprises the one or more gold metal binding peptides and/or one or more combined gold metal binding and reducing peptides are selected from the group consisting of SEQ ID NO:1, SEQ ID NO:2, and SEQ ID NO: 28. In some embodiments, the one or more gold metal binding peptides in the nanoparticle or nanoparticle composition have at least 70% sequence identity to SEQ ID NO: 1. In some embodiments, the one or more gold metal binding peptides in the nanoparticle or nanoparticle composition have 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 1. In some embodiments, the one or more gold metal binding peptides in the nanoparticle or nanoparticle composition comprises SEQ ID NO: 1.

In some embodiments, the one or more combined gold metal binding and reducing peptides in the nanoparticle or nanoparticle composition have at least 70% sequence identity to SEQ ID NO: 2. In some embodiments, the one or more combined gold metal binding and reducing peptides in the nanoparticle or nanoparticle composition have 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 2. In some embodiments, the one or more combined gold metal binding and reducing peptides in the nanoparticle or nanoparticle composition comprises SEQ ID NO: 2.

In some embodiments, the one or more combined gold metal binding peptide and the gold reducing peptide have at least 70% sequence identity to SEQ ID NO: 28. In some embodiments, the one or more combined gold metal binding peptide and the gold reducing peptide has 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 28. In some embodiments, the one or more combined gold metal binding peptide and the gold reducing peptide comprises SEQ ID NO: 28.

In some embodiments, the nanoparticle or nanoparticle composition comprises the one or more silver metal binding peptides selected from the group consisting of SEQ ID NO: 7 and SEQ ID NO: 27. In some embodiments, one of the one or more metals is silver and the composition comprising the ion is silver nitrate (AgNO₃). In some embodiments, the one or more silver metal binding peptides have at least 70% sequence identity to SEQ ID NO: 7. In some embodiments, the one or more silver metal binding peptides have 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 7. In some embodiments, the one or more silver metal binding peptides comprises SEQ ID NO: 7.

In some embodiments, the one or more silver metal binding peptides have at least 70% sequence identity to SEQ ID NO: 27. In some embodiments, the one or more silver metal binding peptides have 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NO: 27. In some embodiments, the one or more silver metal binding peptides comprise SEQ ID NO: 27.

In some embodiments, the nanoparticle or nanoparticle composition comprises at least one metal reducing peptide. In some embodiments, the metal reducing peptide comprises any combination tryptophan and/or histidine amino acids. In some embodiments, the nanoparticle or nanoparticle composition comprises at least one tryptophan amino acid. In some embodiments, the metal reducing peptide in the nanoparticle or nanoparticle composition comprises 1, 2, 3, 4, 5, 6, 7, 8, 9, 10, 11, 12, 13, 14, 15, or more tryptophan amino acids. In some embodiments, the metal reducing peptide in the nanoparticle or nanoparticle composition comprises WW. In some embodiments, the metal reducing peptide in the nanoparticle or nanoparticle composition comprises WWW. In some embodiments, the metal reducing peptide in the nanoparticle or nanoparticle composition comprises WWWW (SEQ ID NO: 3). In some embodiments, the metal reducing peptide in the nanoparticle or nanoparticle composition comprises WWWWW (SEQ ID NO: 4). In some embodiments, the metal reducing peptide in the nanoparticle or nanoparticle composition comprises WWWWWW (SEQ ID NO: 5). In some embodiments, the metal reducing peptide in the nanoparticle or nanoparticle composition comprises WWWWWWW (SEQ ID NO: 6). In some embodiments, the metal reducing peptide in the nanoparticle or nanoparticle composition comprises at least one histidine amino acid. In some embodiments, the metal reducing peptide in the nanoparticle or nanoparticle composition comprises 1, 2, 3, 4, 5, 6, 7, 8, 9, 10, 11, 12, 13, 14, 15, or more histidine amino acids.

In some embodiments the nanoparticle or nanoparticle composition comprises two or more copies of SEQ ID NO:1, SEQ ID NO:2, SEQ ID NO:7, SEQ ID NO: 27, and/or SEQ ID NO: 28.

In some embodiments, the nanoparticle or nanoparticle composition comprises one or more linkers. In some embodiments, the nanoparticle or nanoparticle composition comprises 1, 2, 3, 4, 5, 6, 7, 8, 9, 10, 11, 12, 13, 14, 15, 16, 17, 18, 19, 20, or more linkers. In some embodiments, the linker comprises SEQ ID NO:8, or a fragment thereof. In some embodiments, the nanoparticle or nanoparticle composition comprises 50% of SEQ ID NO: 8. In some embodiments, the nanoparticle or nanoparticle composition comprises 1, 2, 3, 4, 5, 6, 7, 8, 9, 10, 11, 12, 13, 14, 15, 16, 17, 18, 19, 20, or more repeats of SEQ ID NO:8.

In some embodiments, the nanoparticle or nanoparticle composition comprises any combination of metal binding peptides of any preceding aspect, metal reducing peptides of any preceding aspect, and linker of any preceding aspect needed to produce the desired effect. Non-limiting examples includes a nanoparticle or nanoparticle composition comprising a WWW-linker-AuBP, AuBP1-linker-WWW, WWW-linker-Midas2, or Midas2-linker-WWW.

In some embodiments, the nanoparticle or nanoparticle composition of any preceding aspect comprises SEQ ID NO: 19, or a variant thereof. In some embodiments, the imaging agent of any preceding aspect is used in vivo as a reporter.

In some embodiments, the nanoparticle or nanoparticle composition further comprises one or more cage-like peptides, or a fragment thereof. In some embodiments, the nanoparticle or nanoparticle composition comprises 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, or more cage-like peptides.

In some embodiments the nanoparticle or nanoparticle composition comprises a cage-like peptide derived from a bacterial species, including but not limited to Thermotoga maritima (T. maritima) and Myxococcus xanthus (M. xanthus). Herein, it is contemplated that the cage-like peptide can alternatively be derived from other bacterial species to synthesize size constrained nanoparticles.

In some embodiments, the nanoparticle or nanoparticle composition comprises an encapsulin cage-like peptide. In some embodiments, the encapsulin cage-like peptide is derived from a bacterial species of any preceding aspect.

The present disclosure also provides a nanoparticle or nanoparticle composition made accordingly to the methods described herein linked to targeting molecule. In some embodiments, the targeting molecule is linked to the metal ion. In some embodiments, the targeting molecule directs the nanoparticle to a specific intracellular compartment, including but not limited to the nucleus, the mitochondria, or the cellular membrane. In some embodiments, the targeting molecule comprises a nuclear localization signal (NLS), a mitochondrial localization signal (MLS), a mitochondrial targeting sequence (MTS), a membrane targeting sequence, or combinations thereof. In some embodiments, the targeting molecule comprises a therapeutic agent. In some embodiments, the targeting molecule comprises an antibody, an inhibitor, an aptamer, an immunotherapeutic agent (including, but not limited to a PD-1 antibody, a PD-L1 antibody, a CTLA-4 antibody), an immune checkpoint inhibitor, a vaccine, or other biological compositions used to treat or prevent diseases.

In some embodiments, the nanoparticle or nanoparticle composition further comprises an imaging agent including, but not limited to 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy 492/515; Bodipy 493/503; Bodipy 500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyde Induced Fluorescence); FITC; Flazo Orange; Fluo-3; Fluo-4; Fluorescein Fluoro-Emerald; Fluoro-Gold (FITC); Fluorescein Diacetate; (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes), or a combination thereof.

A number of embodiments of the disclosure have been described. Nevertheless, it will be understood that various modifications may be made without departing from the spirit and scope of the invention. Accordingly, other embodiments are within the scope of the following claims. By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1

### Methods for Genetically Encoded Synthesis of Metal Nanoparticles in Mammalian Cells

### A. Preparation of the Plasmids:

The plasmids encoding the gene sequences for Green Fluorescent Protein (GFP), linkers (short sequences of nucleotides/amino acids that link two functional domains and provide flexibility), various protein cages and metal ion binding and reducing peptides were synthesized by Epoch Life Science. Two basic genetically encoded metal nanoparticle synthesis processes were adopted. In the first process, encapsulin protein cages of bacteria T. maritima and M. xanthus were engineered to express silver and gold binding and reducing peptides instead of the native ferritin-like protein. In the second process, gold ion binding and reducing peptides AuBP and Midas were used. The encapsulin protein cages (with gold and silver binding and reducing peptides) and the Midas and AuBP gold binding peptides when exposed to Silver Nitrate (AgNO₃) and Gold Chloride Trihydrate (HAuCl₄) produced size constrained silver and gold nanoparticles respectively. The amino acid sequences of the constructs used are as follows.

### AuBP and Midas Gold Binding Peptide Sequences:

1. 2W-AuBP1-Linker3a-GFP:
2. 3W-AuBP1-Linker3a-GFP:
3. GFP-Linker3a-2W-AuBP1:
4. GFP-Linker3a-3W-AuBP1:
5. MIDAS2-Linker3a-GFP:
6. GFP-Linker3a-MIDAS2:
   T. maritima Encapsulin with Silver Binding Peptide Sequences:
7. M-AG4-Linker2-EncTM_Residue 1-138-GGTS-GFP-GG-EncTM_Residue 139-End:
   M. xanthus Encapsulin with Silver Binding Peptide Sequences:
8. AG4-Linker3a-EncSig-Linker3a-EncA-Linker3b-GFP
   T. maritima Encapsulin with Gold Binding Peptide Sequences:
9. M-A3-Linker2-EncTM_Residue 1-138-GGTS-GFP-GG-EncTM_Residue 139-End
   M. xanthus Encapsulin with Gold Binding Peptide Sequences:
10. A3-Linker3a-EncSig-Linker3a-EncA-Linker3b-GFP

### B. Transfection, Salt Addition and Imaging:

The plasmids containing the coding DNA sequences of the proteins/peptides mentioned above were augmented using competent E. coli and were isolated from the bacteria using Genejet plasmid miniprep kit (Thermo Scientific) following standard protocols from the manufacturers. HeLa and HMC3 (Microglia) cells used were bought from ATCC. The cells were transfected with suitable amounts of plasmid using Lipofectamine 2000 and 3000 (Thermo Scientific). The transfection leading to the expression of the proteins/peptides was verified by GFP signals (encoded in the gene sequence) from the transfected cells. The cells were imaged using EVOS M5000 fluorescent and Nikon Eclipse confocal microscopes. The amount of plasmid, transfection reagent and incubation times required for best transfection of the cells (approximately 80%) were calculated based on the results of screening experiments performed using various quantities of the reagents and the plasmids.

Gold (III) Chloride Trihydrate (HAuCl4) and Silver Nitrate (AgNO3) were bought from Sigma Aldrich and Fisher Chemical respectively. Suitable amounts of HAuCl4 and AgNO3 were added to 1×PBS and 50 mM HEPES with continuous stirring until the salts dissolved. The solutions were freshly prepared for each experiment and stored in tubes wrapped in aluminum foil to protect from light. The salt solutions were added to transfected cells (expressing GFP) and non-transfected cells (used as controls). Other controls used were cells cultured in media and cells transfected with the plasmids without the addition of the salts. After salt addition, the health of the cells was monitored at regular intervals using the light and confocal microscopes. After addition of the salts, the cells were incubated for varying amounts of time at 37 degree C. and 5% CO2 followed by imaging and fixation with 2.5% glutaraldehyde to prepare them for processing for Transmission Electron Microscope imaging.

After fixing with glutaraldehyde the samples were processed using standard Transmission Electron Microscope (TEM) sample processing procedure and mounted on copper grids for imaging. JEOL 1400-PLUS 120 kV TEM at the Center for Biologic Imaging at the University of Pittsburgh was used for imaging the samples. The images were obtained at various magnifications as found suitable. Scanning Electron Microscopy with Energy Dispersive X-ray Spectroscopy (SEM-EDS) of the processed samples were performed using ZEISS Sigma 500VP SEM at the Nanoparticle Fabrication and Characterization Facility at University of Pittsburgh.

### Example 2

### Description of Genetically Encoded Nanoparticles

To transform nanoparticles into useful biomedical tools, living cells are engineered to synthesize nanoparticles on their own. Cells are presented with subtoxic concentrations of metals, as ions that will form the nanoparticle of interest, and will drive the formation of nanoparticles through expression of proteins that will cage or reduce these ions. By having the nanoparticles-of-interest genetically encoded, the aggregation and targeting problems are solved. The aggregation challenge is solved through the place of nanoparticle production and the physical separation of the sites of their production, while the targeting challenge is solved by using molecular ‘zip code’ sequences to transport the nanoparticles into the intracellular target of interest. Having the nanoparticles genetically encoded unlocks a plethora of biomedical applications. To demonstrate the strength of the new technology, radiation resistance, antimicrobial activity, and anticancer therapy will be the focus of treatment.

For radiation resistance, cells are protected against UV radiation, which causes cancer, and ionizing radiation, which can cause severe radiation sickness, rapid death, and cancer. To make the mammalian tissue radiation-resistant, cage-like proteins apoferritin or encapsuling are used as nanoparticle forming strategies. For UV resistance, light scattering ZnO or TiO2 nanoparticles are used to produce light. For ionizing radiation resistance, heavy metal quantum dots, such as lead sulfide (PbS), which is insoluble and non-toxic in physiological conditions are used and targeted to the nucleus to protect cells' DNA from damage. Second, antibacterial nanoparticles, such as silver nanoparticles (AgNPs), are created. To produce AgNPs in the time and place of interest, the protein silk-sericin, which will reduce AgNO₃, is expressed to produce elemental silver nanoparticles. This elemental silver is used to lyse drug-resistant strains of Salmonella, demonstrating the ability of genetically encoded AgNPs to prevent incurable diseases. Last, genetically encoded anticancer nanoparticle therapy is produced. Genetically encoded cages are used to produce gold nanoparticles inside cancer cells, followed by photothermal therapy to destroy these cells. Silver nanoparticles are produced inside cancer cells to prevent the formation of tumor-feeding blood vessels. This demonstrates the development of a new field of genetic nanotechnology, to treat disease and improve human health.

### Example 3

### Features of Genetically Encoded Nanoparticles

In some embodiments, inorganic nanoparticles are produced synthetically. In order to be administered into the tissue of interest, nanoparticles need to be functionalized of modified chemically, mainly because of potential aggregation of the nanoparticles and nanoparticle mis-targeting to a tissue, a cell or an organelle. The delivery of nanoparticles to the tissue, cell and organelle of interest demand a careful control and design of the nanoparticles, through their size, elasticity, surface modification and shape. To mitigate the targeting and aggregation issues, several nanoparticle delivery strategies have been developed. Among them (a) coating the nanoparticles with lipids; (b) electroporation, through which the cell membranes are disrupted to form pores through which nanoparticles can pass; (c) Intracellular injection of nanoparticles, to cross the membrane barrier and enter into the cells cytoplasm. The above strategies can be low-yield and cumbersome. Even once nanoparticles reach their target cell, another issue that arises in conventional nanotechnology is intracellular mis-targeting. When a nanoparticle is targeted to a cell of interest, it is likely to be endocytosed. The nanoparticles can stay trapped in an endosome, which gradually acidify, or the endosome can fuse with lysosomes that contain enzymes, which might degrade the nanoparticle coatings. This entrapment can hinder the nanoparticles from reaching any non-endosomal intracellular target. To mitigate the endosomal entrapment problem, nanoparticles can be coated with endosomal escape molecules, such as cell-penetrating peptides that cause release from an endosome, rupture of the endosomal membrane through with membrane disrupting polymers, and protein sequences that can be cleaved by endosomal enzymes. A major factor contributing for mis-targeting of nanoparticles, is the instantaneous coating by a ‘protein-corona’ immediately after tissue administration. This protein coat changes the effective size and the surface properties of the nanoparticle, completely altering the biological properties of nanoparticles compared to their original synthetic properties. To mitigate the effect of the protein corona, nanoparticles or the vehicles carrying them are coated with a recognition molecule such as an antibody or an aptamer. This strategy was used to increase anti-tumor nanoparticle internalization into cells. Another anti-corona strategy was to coat the nanoparticle with positively zwitterionic functional groups, which form electrostatic interactions with water molecules, stabilize the nanoparticle and decrease the formation of a protein corona.

Unlike the conventional nanoparticles that are first synthesized and then modified to increase the chance of delivery, disclosed herein are nanoparticles of interest that are genetically encoded. Genetically encoded ‘nanoscale reactors’ or ‘nanoscale cages’ proteins are used to stabilize or reduce metal ions, to produce nanoparticles. These ‘nanoparticle forming genes’ are expressed, then the cells are presented with subtoxic concentrations of a ‘nanoparticle precursor’ which is a salt containing metal ions, which will diffuse into the cells and reduced by the metal reducing peptide inside the cage, forming the of the nanoparticle of interest inside the cage. The ability to produce nanoparticles inside cells enables expressing monodisperse nanoparticles in the time and place of interest. With genetically encoded nanoparticles, there is no need to target the nanoparticle into the tissue or cell of interest using advanced chemistry. Instead, one can express the protein that forms the nanoparticle. Fortunately, there are multiple well-established strategies to perform gene delivery, such as using viruses, Modified RNAs, lipid polymers and amino acid polymers. For comparison, in conventional nanotechnology, targeting of nanoparticles to a given organelle in a given cell in a given tissue will demand four steps: nanoparticle production, modification of nanoparticles to achieve solubility in aqueous solution, coating with cell targeting groups and coating the nanoparticle with endosomal escape/organelle targeting surface groups. In contrast, this genetically encoded strategy offers two steps: delivery of a nanoparticle forming genes, followed by administration of the nanoparticle precursor. This strategy is more effective, faster, cheaper, and less toxic and therefore, more applicable.

### Example 4

### Exemplary Constructs.

1. Linker3a-7WAuBP1
2. Linker3a-3WAuBP1-Linker3a-3WAuBP1-Linker3a-3WAuBP1
3. Linker3a-Midas2-Linker3a-Midas2-Linker3a-Midas2
4. Linker3a-3WAuBP1-Linker3a-Midas2-Linker3a-3WAuBP1-Linker3a-Midas2-Linker3a-3WAuBP1-Linker3a-Midas2
5. Linker3a-AgBP2
6. AgBP2-Linker3a
7. Linker3a-AgBP2
8. AgBP2-Linker3a
9. Linker3a-3WAuBP1-Linker3a-AgBP2
10. Linker3a-Midas2-Linker3a-AgBP2

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

SEQUENCES:

1. SEQ ID NO: 1-AuBP1 metal binding peptide
AGAKRLVLRRE
2. SEQ ID NO: 2-Midas2 gold metal binding peptide
TGTSVLIATPYV
3. SEQ ID NO: 3-Reducing peptide
WWWW
4. SEQ ID NO: 4-Reducing peptide
WWWWW
5. SEQ ID NO: 5-Reducing peptide
WWWWWW
6. SEQ ID NO: 6-Reducing peptide
WWWWWWW
7. SEQ ID NO: 7-Ag4 metal binding peptide
NPSSLFRYLPSD
8. SEQ ID NO: 8-Linker3a
GGGGSGGGGS
9. SEQ ID NO: 9-2W-AuBP1-Linker3a-GFP
WWAGAKRLVLRREGGGGSGGGGSMSKGEELFTGVVPILVELDGDVNGHKFSVSGEGEGD
ATYGKLTLKFICTTGKLPVPWPTLVTTFSYGVQCFSRYPDHMKQHDFFKSAMPEGYVQER
TIFFKDDGNYKTRAEVKFEGDTLVNRIELKGIDFKEDGNILGHKLEYNYNSHNVYIMADKQ
KNGIKVNFKIRHNIEDGSVQLADHYQQNTPIGDGPVLLPDNHYLSTQSALSKDPNEKRDHM
VLLEFVTAAGITHGMDELYK
10. SEQ ID NO: 10-3W-AuBP1-Linker3a-GFP
WWWAGAKRLVLRREGGGGSGGGGSMSKGEELFTGVVPILVELDGDVNGHKFSVSGEGE
GDATYGKLTLKFICTTGKLPVPWPTLVTTFSYGVQCFSRYPDHMKQHDFFKSAMPEGYVQ
ERTIFFKDDGNYKTRAEVKFEGDTLVNRIELKGIDFKEDGNILGHKLEYNYNSHNVYIMAD
KQKNGIKVNFKIRHNIEDGSVQLADHYQQNTPIGDGPVLLPDNHYLSTQSALSKDPNEKRD
HMVLLEFVTAAGITHGMDELYK
11. SEQ ID NO: 11-GFP-Linker3a-2W-AuBP1
MSKGEELFTGVVPILVELDGDVNGHKFSVSGEGEGDATYGKLTLKFICTTGKLPVPWPTLV
TTFSYGVQCFSRYPDHMKQHDFFKSAMPEGYVQERTIFFKDDGNYKTRAEVKFEGDTLVN
RIELKGIDFKEDGNILGHKLEYNYNSHNVYIMADKQKNGIKVNFKIRHNIEDGSVQLADHY
QQNTPIGDGPVLLPDNHYLSTQSALSKDPNEKRDHMVLLEFVTAAGITHGMDELYKGGGG
SGGGGSWWAGAKRLVLRRE
12. SEQ ID NO: 12-GFP-Linker3a-3W-AuBP1
MSKGEELFTGVVPILVELDGDVNGHKFSVSGEGEGDATYGKLTLKFICTTGKLPVPWPTLV
TTFSYGVQCFSRYPDHMKQHDFFKSAMPEGYVQERTIFFKDDGNYKTRAEVKFEGDTLVN
RIELKGIDFKEDGNILGHKLEYNYNSHNVYIMADKQKNGIKVNFKIRHNIEDGSVQLADHY
QQNTPIGDGPVLLPDNHYLSTQSALSKDPNEKRDHMVLLEFVTAAGITHGMDELYKGGGG
SGGGGSWWWAGAKRLVLRRE
13. SEQ ID NO: 13-MIDAS2-Linker3a-GFP
TGTSVLIATPYVGGGGSGGGGSMSKGEELFTGVVPILVELDGDVNGHKFSVSGEGEGDAT
YGKLTLKFICTTGKLPVPWPTLVTTFSYGVQCFSRYPDHMKQHDFFKSAMPEGYVQERTIF
FKDDGNYKTRAEVKFEGDTLVNRIELKGIDFKEDGNILGHKLEYNYNSHNVYIMADKQKN
GIKVNFKIRHNIEDGSVQLADHYQQNTPIGDGPVLLPDNHYLSTQSALSKDPNEKRDHMVL
LEFVTAAGITHGMDELYK
14. SEQ ID NO: 14-GFP-Linker3a-MIDAS2
MSKGEELFTGVVPILVELDGDVNGHKFSVSGEGEGDATYGKLTLKFICTTGKLPVPWPTLV
TTFSYGVQCFSRYPDHMKQHDFFKSAMPEGYVQERTIFFKDDGNYKTRAEVKFEGDTLVN
RIELKGIDFKEDGNILGHKLEYNYNSHNVYIMADKQKNGIKVNFKIRHNIEDGSVQLADHY
QQNTPIGDGPVLLPDNHYLSTQSALSKDPNEKRDHMVLLEFVTAAGITHGMDELYKGGGG
SGGGGSTGTSVLIATPYV
15. SEQ ID NO: 15-T. maritima Encapsulin with silver binding peptide sequences; M-AG4-
Linker2-EncTM_Residue 1-138-GGTS-GFP-GG-EncTM_Residue 139-End
MNPSSLFRYLPSDGGMEFLKRSFAPLTEKQWQEIDNRAREIFKTQLYGRKFVDVEGPYGWE
YAAHPLGEVEVLSDENEVVKWGLRKSLPLIELRATFTLDLWELDNLERGKPNVDLSSLEET
VRKVAEFEDEVIFRGCEKSGVKGLLSFEERKGGTSMSKGEELFTGVVPILVELDGDVNGHK
FSVSGEGEGDATYGKLTLKFICTTGKLPVPWPTLVTTFSYGVQCFSRYPDHMKQHDFFKSA
MPEGYVQERTIFFKDDGNYKTRAEVKFEGDTLVNRIELKGIDFKEDGNILGHKLEYNYNSH
NVYIMADKQKNGIKVNFKIRHNIEDGSVQLADHYQQNTPIGDGPVLLPDNHYLSTQSALSK
DPNEKRDHMVLLEFVTAAGITHGMDELYKGGIECGSTPKDLLEAIVRALSIFSKDGIEGPYT
LVINTDRWINFLKEEAGHYPLEKRVEECLRGGKIITTPRIEDALVVSERGGDFKLILGQDLSI
GYEDREKDAVRLFITETFTFQVVNPEALILLKF
16. SEQ ID NO: 16-M. xanthus Encapsulin with silver binding peptide sequences; AG4-
Linker3a-EncSig-Linker3a-EncA-Linker3b-GFP
NPSSLFRYLPSDGGGGSGGGGSLTVGSLRRGGGGSGGGGSMPLEPHFMPDFLGHAENPLRE
EEWARLNETVIQVARRSLVGRRILDIYGPLGAGVQTVPYDEFQGVSPGAVDIVGEQETAMV
FTDARKFKTIPIIYKDFLLHWRDIEAARTHNMPLDVSAAAGAAALCAQQEDELIFYGDARL
GYEGLMTANGRLTVPLGDWTSPGGGFQAIVEATRKLNEQGHFGPYAVVLSPRLYSQLHRI
YEKTGVLEIETIRQLASDGVYQSNRLRGESGVVVSTGRENMDLAVSMDMVAAYLGASRM
NHPFRVLEALLLRIKHPDAICTLEGAGATERRGSGMSKGEELFTGVVPILVELDGDVNGHK
FSVSGEGEGDATYGKLTLKFICTTGKLPVPWPTLVTTFSYGVQCFSRYPDHMKQHDFFKSA
MPEGYVQERTIFFKDDGNYKTRAEVKFEGDTLVNRIELKGIDFKEDGNILGHKLEYNYNSH
NVYIMADKQKNGIKVNFKIRHNIEDGSVQLADHYQQNTPIGDGPVLLPDNHYLSTQSALSK
DPNEKRDHMVLLEFVTAAGITHGMDELYK
17. SEQ ID NO: 17-T. maritima Encapsulin with gold binding peptide sequences; M-A3-
Linker2-EncTM_Residue 1-138-GGTS-GFP-GG-EncTM_Residue 139-End
MAYSSGAPPMPPFGGMEFLKRSFAPLTEKQWQEIDNRAREIFKTQLYGRKFVDVEGPYGW
EYAAHPLGEVEVLSDENEVVKWGLRKSLPLIELRATFTLDLWELDNLERGKPNVDLSSLEE
TVRKVAEFEDEVIFRGCEKSGVKGLLSFEERKGGTSMSKGEELFTGVVPILVELDGDVNGH
KFSVSGEGEGDATYGKLTLKFICTTGKLPVPWPTLVTTFSYGVQCFSRYPDHMKQHDFFKS
AMPEGYVQERTIFFKDDGNYKTRAEVKFEGDTLVNRIELKGIDFKEDGNILGHKLEYNYNS
HNVYIMADKQKNGIKVNFKIRHNIEDGSVQLADHYQQNTPIGDGPVLLPDNHYLSTQSALS
KDPNEKRDHMVLLEFVTAAGITHGMDELYKGGIECGSTPKDLLEAIVRALSIFSKDGIEGPY
TLVINTDRWINFLKEEAGHYPLEKRVEECLRGGKIITTPRIEDALVVSERGGDFKLILGQDLS
IGYEDREKDA VRLFITETFTFQVVNPEALILLKF
18. SEQ ID NO: 18-M. xanthus Encapsulin with gold binding peptide sequences; A3-Linker3a-
EncSig-Linker3a-EncA-Linker3b-GFP
AYSSGAPPMPPFGGGGSGGGGSLTVGSLRRGGGGSGGGGSMPLEPHFMPDFLGHAENPLR
EEEWARLNETVIQVARRSLVGRRILDIYGPLGAGVQTVPYDEFQGVSPGAVDIVGEQETAM
VFTDARKFKTIPIIYKDFLLHWRDIEAARTHNMPLDVSAAAGAAALCAQQEDELIFYGDAR
LGYEGLMTANGRLTVPLGDWTSPGGGFQAIVEATRKLNEQGHFGPYAVVLSPRLYSQLHR
IYEKTGVLEIETIRQLASDGVYQSNRLRGESGVVVSTGRENMDLAVSMDMVAAYLGASRM
NHPFRVLEALLLRIKHPDAICTLEGAGATERRGSGMSKGEELFTGVVPILVELDGDVNGHK
FSVSGEGEGDATYGKLTLKFICTTGKLPVPWPTLVTTFSYGVQCFSRYPDHMKQHDFFKSA
MPEGYVQERTIFFKDDGNYKTRAEVKFEGDTLVNRIELKGIDFKEDGNILGHKLEYNYNSH
NVYIMADKQKNGIKVNFKIRHNIEDGSVQLADHYQQNTPIGDGPVLLPDNHYLSTQSALSK
DPNEKRDHMVLLEFVTAAGITHGMDELYK
19. SEQ ID NO: 19-Green Fluorescent Protein (GFP).
MVSKGEELFTGVVPILVELDGDVNGHKFSVSGEGEGDATYGKLTLKFICTTGKLPVPWPTL
VTTFSYGVQCFSRYPDHMKQHDFFKSAMPEGYVQERTIFFKDDGNYKTRAEVKFEGDTLV
NRIELKGIDFKEDGNILGHKLEYNYNSHNVYIMADKQKNGIKVNFKIRHNIEDGSVQLADH
YQQNTPIGDGPVLLPDNHYLSTQSALSKDPNEKRDHMVLLEFVTAAGITHGMDELYK
20. SEQ ID NO: 20-EncTM_Residue 1-138 aa
MEFLKRSFAPLTEKQWQEIDNRAREIFKTQLYGRKFVDVEGPYGWEYAAHPLGEVEVLSD
ENEVVKWGLRKSLPLIELRATFTLDLWELDNLERGKPNVDLSSLEETVRKVAEFEDEVIFR
GCEKSGVKGLLSFEERK
21. SEQ ID NO: 21-EncTM_Residue 139-End aa
IECGSTPKDLLEAIVRALSIFSKDGIEGPYTL VINTDRWINFLKEEAGHYPLEKRVEECLRGG
KIITTPRIEDALVVSERGGDFKLILGQDLSIGYEDREKDAVRLFITETFTFQVVNPEALILLKF
22. SEQ ID NO: 22-GGTS Linker
GGTS
23. SEQ ID NO: 23-EncSig
LTVGSLRR
24. SEQ ID NO: 24-EncA
MPLEPHFMPDFLGHAENPLREEEWARLNETVIQVARRSLVGRRILDIYGPLGAGVQTVPYD
EFQGVSPGAVDIVGEQETAMVFTDARKFKTIPIIYKDFLLHWRDIEAARTHNMPLDVSAAA
GAAALCAQQEDELIFYGDARLGYEGLMTANGRLTVPLGDWTSPGGGFQAIVEATRKLNEQ
GHFGPYAVVLSPRLYSQLHRIYEKTGVLEIETIRQLASDGVYQSNRLRGESGVVVSTGREN
MDLAVSMDMVAAYLGASRMNHPFRVLEALLLRIKHPDAICTLEGAGATERR
25. SEQ ID NO: 25-Linker3B
GSG
26. SEQ ID NO: 26-A3
AYSSGAPPMPPF
27. SEQ ID NO: 27-AgBP2
EQLGVRKELRGV
28. SEQ ID NO: 28-D4 or GOLD NECKLACE
GGGGSGGGGSWWWAGAKRLVLRREGGGGSGGGGSTGTSVLIATPYVGGGGSGGGGSW
WWAGAKRLVLRREGGGGSGGGGSTGTSVLIATPYVGGGGSGGGGSWWWAGAKRLVLR
REGGGGSGGGGSTGTSVLIATPYV

1. A method of making a nanoparticle comprising one or more metals in a cell, the method comprising:
a) expressing one or more nucleic acid sequences encoding one or more metal binding peptides and one or more metal reducing peptides and/or one or more combined metal binding and reducing peptides in the cell;
b) administering a composition comprising an ion of each of the one or more metals, or a salt thereof, to the cell; and
c) forming the nanoparticle within the cell upon reduction of the ion of each of the one or more metals.

2. The method of claim 1, wherein the one or more metals are selected from the group consisting of gold, silver, zinc, titanium, lead and potassium.

3. The method of claim 1, wherein the composition comprising the ion of each of the one or more metals is selected from a group consisting of chloroauric acid (HAuCl₄), silver nitrate (AgNO₃), zinc oxide (ZnO), titanium dioxide (TiO₂), lead sulfide (PbS), potassium tetrachloroplatinate (K₂PtCl₄), and any salt thereof.

4. The method of claim 1, wherein one of the one or more metals is gold and the composition comprising the ion is chloroauric acid (HAuCl₄).

5. The method of claim 4, wherein the one or more gold metal binding peptides and/or the one or more combined metal binding and reducing peptides comprise a sequence selected from the group consisting of SEQ ID NO:1, SEQ ID NO:2, and SEQ ID NO: 28.

6. The method of claim 1, wherein the one or more gold metal reducing peptides are selected from the group consisting of WW, WWW, SEQ ID NO:3, SEQ ID NO:4, SEQ ID NO:5, and SEQ ID NO: 6.

7. The method of claim 1, wherein one of the one or more metals is silver and the composition comprising the ion is silver nitrate (AgNO₃).

8. The method of claim 7, wherein the one or more silver metal binding peptides and the one or more silver metal reducing peptides and/or one or more combined silver binding and reducing peptides comprise a sequence selected from the group consisting of SEQ ID NO: 7 and SEQ ID NO: 27.

9.-12. (canceled)

13. The method of claim 1, wherein the nucleic acid sequence further comprises a sequence encoding one or more cage-like peptides, or a fragment thereof.

14. The method of claim 13, wherein the one or more cage-like peptides are selected from the group consisting of apoferritin, encapsulin, sericin, laccase, and ligninase.

15. (canceled)

16. The method of claim 4, wherein the cell is a cancer cell in a subject and the method further comprises administering a therapeutically effective amount of an electromagnetic radiation to the cancer cell, and wherein the method treats the cancer.

17. The method of claim 4, wherein the cell is in a subject and the method further comprises detecting the location of the nanoparticle in the subject using an imaging method selected from computed tomography (CT), positron emission tomography (PET), and magnetic resonance imaging (MRI).

18. The method of claim 4, wherein the cell is in a subject, the subject has a microbial infection, and wherein the method treats the microbial infection.

19. (canceled)

20. (canceled)

21. A nanoparticle composition comprising a metal, one or more metal binding peptides and one or more metal reducing peptides.

22. The composition of claim 21, wherein one or more metals are selected from the group consisting of gold, silver, zinc, titanium, lead and potassium.

23. (canceled)

24. The composition of claim 22, wherein the one or more gold metal binding peptides and/or one or more combined gold metal binding and reducing peptides comprise a sequence selected from the group consisting of SEQ ID NO:1, SEQ ID NO:2, and SEQ ID NO: 28.

25. The composition of claim 22, wherein the one or more gold metal reducing peptides are selected from the group consisting of WW, WWW, SEQ ID NO: 3, SEQ ID NO:4, SEQ ID NO:5, and SEQ ID NO: 6.

26. (canceled)

27. The composition of claim 22, wherein the one or more silver metal binding peptides and the one or more silver metal reducing peptides and/or one or more combined silver binding and reducing peptides comprise a sequence selected from the group consisting of SEQ ID NO:7 and SEQ ID NO: 27.

28.-31. (canceled)

32. The method of claim 13, further comprising exposing the cell to a radiation, and wherein radiation damage in the cell is reduced compared to a control.

33. The method of claim 32, wherein the cell is in a subject and the radiation is x-rays, gamma rays, electron beams, protons, or combinations thereof.


SEQUENCE LISTING (28 sequences)

SEQ ID NO: 1 - protein, 11 aa, synthetic construct
  REGION 1..11: note = Description of sequence: AuBP1 metal binding peptide
AGAKRLVLRRE

SEQ ID NO: 2 - protein, 12 aa, synthetic construct
  REGION 1..12: note = Description of sequence: Midas2 metal binding peptide
TGTSVLIATPYV

SEQ ID NO: 3 - protein, 4 aa, synthetic construct
  REGION 1..4: note = Description of sequence: reducing peptide
WWWW

SEQ ID NO: 4 - protein, 5 aa, synthetic construct
  REGION 1..5: note = Description of sequence: reducing peptide
WWWWW

SEQ ID NO: 5 - protein, 6 aa, synthetic construct
  REGION 1..6: note = Description of sequence: reducing peptide
WWWWWW

SEQ ID NO: 6 - protein, 7 aa, synthetic construct
  REGION 1..7: note = Description of sequence: reducing peptide
WWWWWWW

SEQ ID NO: 7 - protein, 12 aa, synthetic construct
  REGION 1..12: note = Description of sequence: Ag4 metal binding peptide
NPSSLFRYLPSD

SEQ ID NO: 8 - protein, 10 aa, synthetic construct
  REGION 1..10: note = Description of sequence: Linker 3a
GGGGSGGGGS

SEQ ID NO: 9 - protein, 261 aa, synthetic construct
  REGION 1..261: note = Description of sequence: 2W-AuBP1-Linker3a-GFP
WWAGAKRLVLRREGGGGSGGGGSMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 10 - protein, 262 aa, synthetic construct
  REGION 1..262: note = Description of sequence: 3W-AuBP1-Linker3a-GFP
WWWAGAKRLVLRREGGGGSGGGGSMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 11 - protein, 261 aa, synthetic construct
  REGION 1..261: note = Description of sequence: GFP-Linker3a-2W-AuBP1
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGGGGSGGGGSWWAGAKRLVLRRE

SEQ ID NO: 12 - protein, 262 aa, synthetic construct
  REGION 1..262: note = Description of sequence: GFP-Linker3a-3W-AuBP1
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGGGGSGGGGSWWWAGAKRLVLRRE

SEQ ID NO: 13 - protein, 260 aa, synthetic construct
  REGION 1..260: note = Description of sequence: MIDAS2-Linker3a-GFP
TGTSVLIATPYVGGGGSGGGGSMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 14 - protein, 260 aa, synthetic construct
  REGION 1..260: note = Description of sequence: GFP-Linker3a-MIDAS2
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGGGGSGGGGSTGTSVLIATPYV

SEQ ID NO: 15 - protein, 524 aa, synthetic construct
  REGION 1..524: note = Description of sequence: T. maritima Encapsulin with silver binding peptide sequences
  DOMAIN 1..138: note = M-AG4-Linker2-EncTM_Residue
  DOMAIN 139..524: note = GGTS-GFP-GG-EncTM_Residue
MNPSSLFRYLPSDGGMEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKGGTSMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGGIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKF

SEQ ID NO: 16 - protein, 575 aa, synthetic construct
  REGION 1..575: note = Description of sequence: M. xanthus Encapsulin with silver binding peptide sequences
NPSSLFRYLPSDGGGGSGGGGSLTVGSLRRGGGGSGGGGSMPLEPHFMPDFLGHAENPLREEEWARLNETVIQVARRSLVGRRILDIYGPLGAGVQTVPYDEFQGVSPGAVDIVGEQETAMVFTDARKFKTIPIIYKDFLLHWRDIEAARTHNMPLDVSAAAGAAALCAQQEDELIFYGDARLGYEGLMTANGRLTVPLGDWTSPGGGFQAIVEATRKLNEQGHFGPYAVVLSPRLYSQLHRIYEKTGVLEIETIRQLASDGVYQSNRLRGESGVVVSTGRENMDLAVSMDMVAAYLGASRMNHPFRVLEALLLRIKHPDAICTLEGAGATERRGSGMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 17 - protein, 524 aa, synthetic construct
  REGION 1..524: note = Description of sequence: T. maritima Encapsulin with gold binding peptide sequences
  DOMAIN 1..138: note = M - A3 - Linker2 - EncTM_Residue
  DOMAIN 139..524: note = GGTS - GFP - GG - EncTM_Residue
MAYSSGAPPMPPFGGMEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERKGGTSMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKGGIECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKF

SEQ ID NO: 18 - protein, 575 aa, synthetic construct
  REGION 1..575: note = Description of sequence: M. xanthus Encapsulin with gold binding peptide sequences
AYSSGAPPMPPFGGGGSGGGGSLTVGSLRRGGGGSGGGGSMPLEPHFMPDFLGHAENPLREEEWARLNETVIQVARRSLVGRRILDIYGPLGAGVQTVPYDEFQGVSPGAVDIVGEQETAMVFTDARKFKTIPIIYKDFLLHWRDIEAARTHNMPLDVSAAAGAAALCAQQEDELIFYGDARLGYEGLMTANGRLTVPLGDWTSPGGGFQAIVEATRKLNEQGHFGPYAVVLSPRLYSQLHRIYEKTGVLEIETIRQLASDGVYQSNRLRGESGVVVSTGRENMDLAVSMDMVAAYLGASRMNHPFRVLEALLLRIKHPDAICTLEGAGATERRGSGMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 19 - protein, 238 aa, synthetic construct
  REGION 1..238: note = Description of sequence: Green Fluorescent Protein (GFP)
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 20 - protein, 138 aa, synthetic construct
  REGION 1..138: note = Description of sequence: EncTM_Residue 1-138 aa
MEFLKRSFAPLTEKQWQEIDNRAREIFKTQLYGRKFVDVEGPYGWEYAAHPLGEVEVLSDENEVVKWGLRKSLPLIELRATFTLDLWELDNLERGKPNVDLSSLEETVRKVAEFEDEVIFRGCEKSGVKGLLSFEERK

SEQ ID NO: 21 - protein, 127 aa, synthetic construct
  REGION 1..127: note = Description of sequence: EncTM_Residue 139-End aa
IECGSTPKDLLEAIVRALSIFSKDGIEGPYTLVINTDRWINFLKEEAGHYPLEKRVEECLRGGKIITTPRIEDALVVSERGGDFKLILGQDLSIGYEDREKDAVRLFITETFTFQVVNPEALILLKF

SEQ ID NO: 22 - protein, 4 aa, synthetic construct
  REGION 1..4: note = Description of sequence: GGTS Linker
GGTS

SEQ ID NO: 23 - protein, 8 aa, synthetic construct
  REGION 1..8: note = Description of sequence: EncSig
LTVGSLRR

SEQ ID NO: 24 - protein, 294 aa, synthetic construct
  REGION 1..294: note = Description of sequence: EncA
MPLEPHFMPDFLGHAENPLREEEWARLNETVIQVARRSLVGRRILDIYGPLGAGVQTVPYDEFQGVSPGAVDIVGEQETAMVFTDARKFKTIPIIYKDFLLHWRDIEAARTHNMPLDVSAAAGAAALCAQQEDELIFYGDARLGYEGLMTANGRLTVPLGDWTSPGGGFQAIVEATRKLNEQGHFGPYAVVLSPRLYSQLHRIYEKTGVLEIETIRQLASDGVYQSNRLRGESGVVVSTGRENMDLAVSMDMVAAYLGASRMNHPFRVLEALLLRIKHPDAICTLEGAGATERR

SEQ ID NO: 25 - skipped

SEQ ID NO: 26 - protein, 12 aa, synthetic construct
  REGION 1..12: note = Description of sequence: A3
AYSSGAPPMPPF

SEQ ID NO: 27 - protein, 12 aa, synthetic construct
  REGION 1..12: note = Description of sequence: AgBP2
EQLGVRKELRGV

SEQ ID NO: 28 - protein, 138 aa, synthetic construct
  REGION 1..138: note = Description of sequence: D4 or GOLD NECKLACE
GGGGSGGGGSWWWAGAKRLVLRREGGGGSGGGGSTGTSVLIATPYVGGGGSGGGGSWWWAGAKRLVLRREGGGGSGGGGSTGTSVLIATPYVGGGGSGGGGSWWWAGAKRLVLRREGGGGSGGGGSTGTSVLIATPYV